(19) United States
(12) Patent Application Publication
(10) Pub. No.: US 2007/0004735 A1
(43) Pub. Date: Jan. 4, 2007

(54) Method for inhibiting the replication of herpes viruses

(76) Inventors: Ulrich Betz, Wuppertal (DE); Guy Hewlett, Wuppertal (DE); Gerald Kleymann, Bad Salzuflen (DE); Thomas Lampe, Dusseldorf (DE); Tse-I Lin, Mechelen (BE); Susanne Nikolic, Monheim (DE); Jurgen Reefshlager, Oldensburg (DE); Tobias Wunberg, Solingen (DE); Holger Zimmerman, Wuppertal (DE); Franz Zumpe, Tonisvorst (DE); Wolfgang Bender, Burscheid (DE); Kerstin Henninger, Wuppertal (DE); Axel Jensen, Velbert (DE); Jorg Keldenich, Wuppertal (DE); Rudolf Schohe-Loop, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/541,886

(22) PCT Filed: Dec. 24, 2003
(86) PCT No.: PCT/EP03/14883

§ 371 Date: Jul. 13, 2006

(30) Foreign Application Priority Data
Jan. 7, 2003 (DE) 10322109.3

(51) Int. Cl. C12Q 1/70; A61K 31/50; A61K 31/17; C07C 271/28

(52) U.S. Cl. 514/247; 435/5; 514/597; 544/239; 564/49; 564/50

(57) ABSTRACT

The invention relates to a method for inhibiting the replication of herpesviruses, to methods for identifying compounds which inhibit the replication of herpesviruses using this method, to compounds having activity against herpesviruses, to methods for their preparation and to their use for producing medicaments for the treatment of herpes infections.

The invention relates to a method for inhibiting the replication of herpesviruses, to methods for identifying compounds which inhibit the replication of herpesviruses using this method, to compounds having activity against herpesviruses, to methods for their preparation and to their use for producing medicaments for the treatment of herpes infections.

The family of herpesviruses is divided into the three subfamilies of alpha-herpesviruses (e.g. herpes simplex virus of type 1 and 2; HSV1 and HSV2), beta-herpesviruses (e.g. cytomegalovirus; HCMV) and gamma-herpesviruses (e.g. Epstein-Barr virus; EBV).

The manifestations of infections with herpes viruses are, depending on the virus type, disorders of various organs such as the skin, the lymphatic system or the central nervous system.

Infections with the beta-herpesvirus HCMV usually occur during childhood and ordinarily have a subclinical course. The proportion of adults infected worldwide is therefore very high (up to 90%, depending on the investigated population).

Within the family of herpesviruses, cytomegalovirus leads to the highest mortality rate among immunocompromised patients. This is attributable to the fact that cytomegaloviruses cause life-threatening generalized disorders, especially pneumonias, in these people.

HCMV infections in pregnant women may result in serious harm to the child.

The virus particles of herpesviruses have diameters of about 150 to 200 nm and are composed of various structural proteins essential for the virus. The virus core—a fibrillary protein matrix with which the double-stranded linear DNA genome is associated—is located in the interior of the particles. The core is surrounded by an icosahedral capsid which consists of 162 capsomers. The major capsid protein (MCP) of human cytomegalovirus is referred to as UL86.

The viral protein UL80 is processed to at least three different proteins which are involved in capsid maturation. The commonest form thereof is the assembly protein AP.

The formation of virus particles—initially B capsids are formed—is controlled by a precursor complex of UL86 and AP which is responsible for translocation of the MCP (UL86) into the cell nucleus. In the cell nucleus, AP assists the formation of structures which form an internal framework within the B capsids. The viral DNA is packaged into the complete B capsids, with AP being ejected from the virus particles. The DNA-containing infectious virus particles are also referred to as C capsids.

No vaccine for prophylaxis of an HCMV infection is currently available. Ganciclovir is mainly employed for the therapy of HCMV infection but causes serious side effects.

A long-existing need for an improved and, in particular, better-tolerated HCMV therapy emerges from this. The demand for an improved HCMV therapy is moreover enhanced by the fact that firstly organ transplantations are increasingly being performed, and additionally there is a continuous increase in the number of people infected with HIV. Complications arising from HCMV reactivation or HCMV infection are possible in both groups of patients owing to the immunosuppression. There is thus an urgent need for an improved HCMV therapy in these cases.

A preferred object of the present application is to indicate a method with which replication of herpesviruses can be inhibited. This is possible through compounds which are targeted at the major capsid protein and moreover inhibit the formation of C capsids, but not of B capsids. Viruses selected for resistence to this compound show one or more mutations in the gene coding for the major capsid protein.

A further object of the present application is to provide a method for identifying compounds having this novel mechanism of action and having activity against herpesviruses.

This object is achieved by a method characterized in that
a) the major capsid protein or one or more fragments of the major capsid protein is/are brought into contact with test compounds, and
b) the binding of the test substances to the major capsid protein or fragments is measured and
c) the compounds which exhibit binding to the major capsid protein or fragments are selected.

In addition, this object can also be achieved by a method characterized in that
a) herpesviruses are brought into contact with test compounds,
b) resistant herpesviruses are selected,
c) the gene coding for the major capsid protein of these resistant herpesviruses is sequenced, and the resulting protein sequence of the major capsid protein is inferred,
d) the compounds with which resistant herpesviruses having one or more amino acid substitutions in the major capsid protein occur are selected.

A method for selecting compounds having activity against herpesviruses means for the purposes of the present invention a method in which compounds which are novel or are known per se are investigated for their activity against herpesviruses.

In this connection, herpesviruses are, for example, beta-herpesviruses, especially the human cytomegalovirus, especially the HCMV strains Ad169 (ATCC VR-538) or Davis (ATCC VR-807). Use of the strain HCMV-Towne (ATCC VR-977) is not preferred because this already harbors a mutation in the UL86 gene (P1189T) which leads to a corresponding resistance to the substances acting by the mechanism of action described herein.

Surprisingly, test compounds which are distinguished by a unique mechanism of action have been found. On cultivation of HCMV under substance pressure, the formation of B capsids is allowed, whereas the formation of infectious C capsids is prevented. Maintenance of the formation of B capsids during the antiviral treatment might represent an advantage in as much as B capsids initially remain present as immunogen during the viral replication cycle, and this might have advantageous effects, for example in the event of a reinvigorated immune system, for a specific immune defense response resulting therefrom.

In the sequence analysis of cytomegaloviruses resistant to the test compounds, surprisingly only mutations in the capsid protein UL86 were found.

Since these mutated viruses are able to grow under substance pressure, it can be concluded therefrom that the test substance acts precisely on this protein in the sensitive wild-type viruses. Electron microscopic and molecular biology investigations show that the substances inhibit the encapsidation of viral DNA and thus the formation of C capsids. There is no impairment of the replication of the viral DNA and the formation of DNA-free immature capsids (B capsids). Compared with the DNA replication inhibitor ganciclovir which is employed clinically as HCMV medicament, no inhibition of DNA synthesis and expression of the late HCMV genes takes place under the influence of the substances of the invention. The mutations found in UL86 occur preferentially in two regions. The first region extends from amino acid position 435 to 689 and the second region from amino acid position 1189 to 1338 (see Table 2).

The antiviral effect may on the one hand arise through direct interaction of the substance with UL86, or else act via an indirect effect on UL86.

Antiviral substances acting by this novel and surprising mechanism of action can also be obtained by further methods such as, for example, molecular modeling with the aid of the three-dimensional structure of a major capsid protein, molecular modeling on the basis of known UL86 inhibitors etc. These methods are well known to the skilled worker.

Little is known about the functions and interactions of UL86 during capsid maturation and DNA packaging (Wood et al., J. Virol, 1997, 71, 179-190).

The anti-HCMV medicaments currently available are not ideal owing to severe side effects. High-throughput testing of large substance libraries has recently led to inhibitors acting on further viral targets (Wathen, Rev Med Virol, 2002, 12, 167-178). The mechanism of action described herein shows a surprising novel option allowing inhibition of the replication of herpesviruses with the aid of compounds.

Major capsid protein-binding compounds can be identified by purification of capsids, recombinant expression of major capsid protein or partial fragments of the major capsid protein and measurement of substances binding to the protein or protein fragment (e.g. HPLC, displacement of fluorescent peptides, displacement of aptamers, various spectroscopic methods etc.), which are well known to the skilled worker.

Identification is additionally possible by the following method with test substances: test compounds mean compounds which are to be investigated for their activity on herpesviruses. These compounds may be novel or known per se. They are brought into contact with the herpesviruses. This preferably takes place by cultivating HCMV in 384-well tissue culture plates. For this purpose, susceptible cells, preferably human fibroblasts, are seeded in tissue culture vessels. Preferably 5×10³ cells are employed per well on a 96-well plate and are infected with HCMV (preferably with an moi of 0.03).

The infections are cultivated with various concentrations of substance (preferably with concentrations from 0.005 to 250 μM) until a distinct CPE is evident in the virus control (usually after 6 days). It is then possible to determine the IC₅₀ from the other concentrations of substance. Active substances are distinguished by an IC₅₀ which is preferably <1 μM and additionally has an SI of >10.

Viruses resistant to active substances can be grown as follows:

HCMV is preferably cultivated again in 96-well tissue culture plates. For this purpose, susceptible cells, preferably human fibroblasts, are seeded in tissue culture vessels. Preferably 5×10³ cells are employed per well on a 96-well plate and are infected with HCMV (preferably with an moi of 0.03). The infections are now cultivated under substance pressure equivalent to 10 times the IC₅₀ of the substance.

Cell cultures which show a cytopathic effect (CPE) comparable with a virus infection without substance pressure are analyzed further, i.e. the viruses are passaged on fresh cell cultures and cultivated further under substance pressure. Viruses which grow further under substance pressure and show an RI (resistance index) of >5 are referred to as resistant viruses.

The DNA of the resistant viruses is isolated and then the nucleotide sequence of the gene coding for the MCP (UL86) is determined and compared with the sequence of the initial virus (wild-type virus which is sensitive to the substance). Resistant viruses which show mutations in the amino acid sequence resulting for the major capsid protein (UL86) identify a substance which can be employed as UL86 inhibitor.

Most screens for identifying anti-HCMV agents make use of the laboratory strain HCMV Towne which has been known for a long time and is well established. Substances acting by the mechanism of action of the invention can be found therewith only with great difficulty or not at all. For this reason, an important method for the therapy and prophylaxis of HCMV infections has to date been completely ignored, but is described in this patent. It is possible in screens for identifying anti-HCMV agents to select substances which inhibit the replication of HCMV Towne only inadequately or not at all but suppress the replication of wild-type HCMV strains. These agents can then be employed for the therapy and prophylaxis of HCMV infections.

Preference is given to

[A] Compounds of the general formula (Ia), 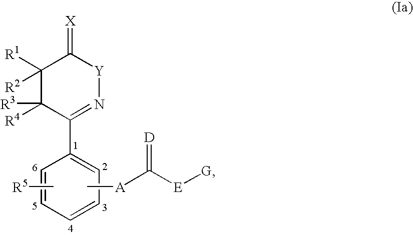
in which
A is linked via positions 2, 3, 5 or 6 to the aromatic system, and
A is oxygen or NR⁶,
E is oxygen, CR⁹R¹⁰ or NR⁷,
Y is oxygen or NR⁸,
D and X are identical or different and are each oxygen or sulfur,
G is hydrogen,
or
G is C₆-C₁₀-aryl, where C₆-C₁₀-aryl may optionally be substituted by up to three substituents selected from the group consisting of halogen, hydroxy, nitro, cyano, C₁-C₆-alkoxy, hydroxycarbonyl, C₁-C₆-alkoxycarbonyl, amino, mono- or di-C₁-C₆-alkylamino, mono- or di-C₁-C₆-alkylaminocarbonyl and C₁-C₆-alkyl,
   in which
   C₁-C₆-alkoxy, C₁-C₆-alkoxycarbonyl, mono- or di-C₁-C₆-alkylamino, mono- or di-C₁-C₆-alkylaminocarbonyl or C₁-C₆-alkyl may optionally be substituted by up to three substituents selected from the group consisting of halogen, hydroxy, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₆-C₁₀-aryl,
or
G is C₆-C₁₀-aryl, where C₆-C₁₀-aryl may optionally be substituted by phenyl,
   in which
   phenyl may optionally be substituted by up to three substituents selected from the group consisting of halogen, hydroxy, C₁-C₆-alkoxy, amino, mono- or di-C₁-C₆-alkylamino, hydroxycarbonyl, C₁-C₆-alkoxycarbonyl, mono- or di-C₁-C₆-alkylaminocarbonyl and C₁-C₆-alkyl,
   in which
   C₁-C₆-alkyl may in turn be optionally substituted by up to three substituents selected from the group consisting of hydroxy, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G is C₆-C₁₀-aryl, where C₆-C₁₀-aryl may optionally be substituted by phenyl,
   in which
   phenyl may optionally be substituted by C₅-C₆-heteroaryl or C₅-C₇-heterocyclyl,
   in which
   C₅-C₆-heteroaryl or C₅-C₇-heterocyclyl may in turn optionally be substituted by up to three substituents selected from the group consisting of halogen, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G is C₆-C₁₀-aryl, where C₆-C₁₀-aryl may optionally be substituted by a group of the following formula 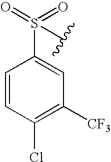
or
G is C₅-C₁₀-heteroaryl or C₅-C₇-heterocyclyl, where C₅-C₁₀-heteroaryl or C₅-C₇-heterocyclyl may optionally be substituted by up to three substituents selected from the group consisting of halogen, nitro, cyano, C₁-C₆-alkyl, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
or
G is C₃-C₁₀-cycloalkyl, where C₃-C₁₀-cycloalkyl may optionally be substituted by up to three substituents selected from the group consisting of halogen, nitro, cyano, hydroxy, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹, R², R³ and R⁴ are identical or different and are each hydrogen, amino, mono- or di-C₁-C₆-alkylamino, C₁-C₆-alkylcarbonylamino, C₆-C₁₀-aryl or C₁-C₆-alkyl, where C₁-C₆-alkyl may optionally be substituted by up to three substituents selected from the group consisting of hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
   and
   where C₆-C₁₀-aryl may optionally be substituted by up to three substituents selected from the group consisting of halogen, hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl, mono- or di-C₁-C₆-alkylaminocarbonyl and C₁-C₆-alkyl,
   in which
   C₁-C₆-alkyl may optionally be substituted by up to three substituents selected from the group consisting of hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R² or R³ and R⁴ form together with the carbon atom to which they are bonded a C₃-C₆-cycloalkyl ring, where the C₃-C₆-cycloalkyl ring may optionally be substituted by up to three substituents selected from the group consisting of halogen, hydroxy, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
or
R¹ and R³ form together with the carbon atoms to which they are bonded a C₃-C₆-cycloalkyl ring, where the C₃-C₆-cycloalkyl ring may optionally be substituted by up to three substituents selected from the group consisting of halogen, hydroxy, C₁-C₆-alkyl, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁵ is hydrogen, halogen, hydroxy, C₁-C₆-alkoxy, amino, mono- or di-C₁-C₆-alkylamino or C₁-C₆-alkyl, where C₁-C₆-alkoxy, mono- or di-C₁-C₆-alkylamino or C₁-C₆-alkyl may optionally be substituted by up to three substituents selected from the group consisting of hydroxy, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ are identical or different and are each hydrogen or C₁-C₆-alkyl, where C₁-C₆-alkyl may optionally be substituted by up to three substituents selected from the group consisting of hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R⁹ and R¹⁰ are identical or different and are each hydrogen, NR¹¹R¹², OR¹³ or C₁-C₆-alkyl, where C₁-C₆-alkyl may optionally be substituted by up to three substituents selected from the group consisting of hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
R¹¹, R¹² and R¹³ are identical or different and are each hydrogen or C₁-C₆-alkyl, where C₁-C₆-alkyl may optionally be substituted by up to three substituents selected from the group consisting of hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
   and the tautomers, stereoisomers, stereoisomeric mixtures thereof and the pharmacologically acceptable salts thereof,
or

[B] compounds of the Formula (Ib) 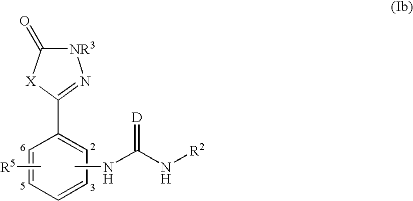
in which
the radical —NHC(D)NHR² is linked via one of positions 2, 3, 5 or 6 to the aromatic system,
X is —N(R⁶)— or a group 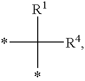
D is oxygen or sulfur,
R¹ is C₆-C₁₀-aryl or C₁-C₆-alkyl, where alkyl may optionally be substituted by up to three substituents independently of one another selected from the group consisting of hydroxy, C₁-C₆-alkoxy, amino, C₁-C₆-alkylamino, C₁-C₆-alkylcarbonylamino, hydroxycarbonyl, C₁-C₆-alkoxycarbonyl and C₁-C₆-alkylaminocarbonyl,
   and
   where aryl may optionally be substituted by up to three substituents independently of one another selected from the group consisting of halogen, hydroxy, C₁-C₆-alkoxy, amino, C₁-C₆-alkylamino, C₁-C₆-alkylcarbonylamino, hydroxycarbonyl, C₁-C₆-alkoxycarbonyl, C₁-C₆-alkylaminocarbonyl and C₁-C₆-alkyl,
   or
R¹ and R⁴ form together with the carbon atom to which they are bonded a C₃-C₆-cycloalkyl ring, where the cycloalkyl ring may optionally be substituted by up to three substituents independently of one another selected from the group consisting of halogen, hydroxy, C₁-C₆-alkyl, C₁-C₆-alkoxy, amino, C₁-C₆-alkylamino, C₁-C₆-alkylcarbonylamino, hydroxycarbonyl, C₁-C₆-alkoxycarbonyl and C₁-C₆-alkylaminocarbonyl,
R² is C₃-C₈-cycloalkyl or C₆-C₁₀-aryl, where aryl may optionally be substituted by up to three substituents independently of one another selected from the group consisting of halogen, hydroxy, nitro, cyano, C₁-C₆-alkoxy, hydroxycarbonyl, C₁-C₆-alkoxycarbonyl, amino, C₁-C₆-alkylamino, C₁-C₆-alkylaminocarbonyl and C₁-C₆-alkyl,
R³ is hydrogen or C₁-C₆-alkyl, where alkyl may optionally be substituted by up to two substituents independently of one another selected from the group consisting of C₁-C₆-alkoxy, hydroxycarbonyl and C₁-C₆-alkoxycarbonyl,
R⁴ is C₁-C₆-alkyl, where alkyl may optionally be substituted by up to three substituents independently of one another selected from the group consisting of hydroxy, C₆-C₁₀-aryl, C₁-C₆-alkoxy, amino, C₁-C₆-alkylamino, C₁-C₆-alkylcarbonylamino, hydroxycarbonyl, C₁-C₆-alkoxycarbonyl and C₁-C₆-alkylaminocarbonyl,
   or
R⁴ is C₆-C₁₀-aryl, where aryl may optionally be substituted by up to three substituents independently of one another selected from the group consisting of halogen, hydroxy, C₁-C₆-alkoxy, amino, C₁-C₆-alkylamino, C₁-C₆-alkylcarbonylamino, hydroxycarbonyl, C₁-C₆-alkoxycarbonyl, C₁-C₆-alkylaminocarbonyl and C₁-C₆-alkyl,
R⁵ is hydrogen, halogen, hydroxy, C₁-C₆-alkoxy, amino, C₁-C₆-alkylamino or C₁-C₆-alkyl,
R⁶ is C₆-C₁₀-aryl, C₃-C₈-cycloalkyl or C₁-C₆-alkyl, where alkyl may optionally be substituted by up to two substituents independently of one another selected from the group consisting of hydroxy, C₆-C₁₀-aryl, C₁-C₆-alkoxy, amino, C₁-C₆-alkylamino, hydroxycarbonyl and C₁-C₆-alkoxycarbonyl,
   and
   where cycloalkyl may optionally be substituted by up to three substituents independently of one another selected from the group consisting of hydroxy, C₁-C₆-alkyl, C₆-C₁₀-aryl, C₁-C₆-alkoxy, amino, C₁-C₆-alkylamino, hydroxycarbonyl and C₁-C₆-alkoxycarbonyl,
      and the tautomers, stereoisomers, stereoisomeric mixtures thereof and the pharmacologically acceptable salts thereof.

Preference is given to compounds of the general formula (Ia) in which
A is linked via positions 2, 3, 5 or 6 to the aromatic system, and
A is NR⁶,
E is NR⁷,
Y is NR⁸,
D and X are oxygen,
G is C₆-C₁₀-aryl, where C₆-C₁₀-aryl may optionally be substituted by up to three substituents which are selected independently of one another from the group consisting of halogen, hydroxy, cyano and C₁-C₆-alkyl,
   in which
   C₁-C₆-alkyl may optionally be substituted by up to three substituents of halogen,
   or
G is C₅-C₆-heteroaryl, where C₅-C₆-heteroaryl may optionally be substituted by up to three substituents which are selected independently of one another from the group consisting of halogen and C₁-C₃-alkyl,
or
G is C₃-C₁₀-cycloalkyl, where C₃-C₁₀-cycloalkyl may optionally be substituted by up to three substituents C₁-C₆-alkyl,
R¹, R² and R³ are identical or different and are each hydrogen or C₁-C₃-alkyl,
R⁴ is hydrogen, C₆-C₁₀-aryl or C₁-C₆-alkyl, where C₁-C₆-alkyl may optionally be substituted by up to three substituents which are selected independently of one another from the group consisting of hydroxy, C₁-C₆-alkoxy, amino, mono- or di-C₁-C₆-alkylamino, C₁-C₆-alkylcarbonylamino, hydroxycarbonyl, C₁-C₆-alkoxycarbonyl and mono- or di-C₁-C₆-alkylaminocarbonyl,
   and
   where C₆-C₁₀-aryl may optionally be substituted by up to three substituents which are selected independently of one another from the group consisting of halogen, hydroxy, C₁-C₆-alkoxy and C₁-C₆-alkyl,
   where R¹, R², R³ and R⁴ are not simultaneously hydrogen,
R⁵ is hydrogen, halogen, hydroxy, amino, mono- or di-C₁-C₆-alkylamino or C₁-C₆-alkyl, where C₁-C₆-alkyl may optionally be substituted by up to three substituents which are selected independently of one another from the group consisting of hydroxy, C₁-C₆-alkoxy, amino, mono- or di-C₁-C₆-alkylamino, hydroxycarbonyl, C₁-C₆-alkoxycarbonyl and mono- or di-C₁-C₆-alkylaminocarbonyl,
R⁶, R⁷ and R⁸ are hydrogen,
or
   compounds of the general formula (Ib) in which
   the radical —NHC(D)NHR² is linked via one of positions 2, 3, 5 or 6 to the aromatic system,
X is —N(R⁶)— or a group 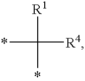
D is oxygen,
R¹ is C₁-C₆-alkyl, where alkyl may optionally be substituted by up to three substituents independently of one another selected from the group consisting of hydroxy, C₁-C₆-alkoxy, amino, C₁-C₆-alkylamino, C₁-C₆-alkylcarbonylamino, hydroxycarbonyl, C₁-C₆-alkoxycarbonyl and C₁-C₆-alkylaminocarbonyl,
or
R¹ and R⁴ form together with the carbon atom to which they are bonded a C₅-C₆-cycloalkyl ring, where the cycloalkyl ring may optionally be substituted by up to three substituents independently of one another selected from the group consisting of halogen, hydroxy, C₁-C₆-alkyl, C₁-C₆-alkoxy, amino, C₁-C₆-alkylamino, C₁-C₆-alkylcarbonylamino, hydroxycarbonyl, C₁-C₆-alkoxycarbonyl and C₁-C₆-alkylaminocarbonyl,
R² is C₆-C₁₀-aryl, where aryl may optionally be substituted by up to three substituents independently of one another selected from the group consisting of halogen or C₁-C₆-alkyl,
R³ is hydrogen or C₁-C₆-alkyl, where alkyl may optionally be substituted by up to two substituents independently of one another selected from the group consisting of C₁-C₆-alkoxy, hydroxycarbonyl and C₁-C₆-alkoxycarbonyl,
R⁴ is C₁-C₆-alkyl, where alkyl may optionally be substituted by up to three substituents independently of one another selected from the group consisting of hydroxy, phenyl, C₁-C₆-alkoxy, amino, C₁-C₆-alkylamino, C₁-C₆-alkylcarbonylamino, hydroxycarbonyl, C₁-C₆-alkoxycarbonyl and C₁-C₆-alkylaminocarbonyl,
R⁵ is hydrogen, halogen, hydroxy, C₁-C₆-alkoxy, amino, C₁-C₆-alkylamino or C₁-C₆-alkyl,
R⁶ is C₃-C₈-cycloalkyl or C₁-C₆-alkyl, where alkyl may optionally be substituted by up to two substituents independently of one another selected from the group consisting of hydroxy, C₆-C₁₀-aryl, C₁-C₆-alkoxy, amino, C₁-C₆-alkylamino, hydroxycarbonyl and C₁-C₆-alkoxycarbonyl,
and
   where cycloalkyl may optionally be substituted by up to three substituents independently of one another selected from the group consisting of C₁-C₆-alkyl and C₁-C₆-alkoxy.

Particular preference is given to

### N-(2,4-Difluorophenyl)-N′-[3-(4,4-dimethyl-6-oxo-1,4,5,6-tetrahydro-3-pyridazinyl)phenyl]-urea

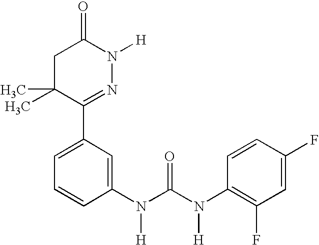

### N-(2,5-Difluorophenyl)-N′-[3-(4,4-dimethyl-6-oxo-1,4,5,6-tetrahydro-3-pyridazinyl)-4-hydroxyphenyl]urea

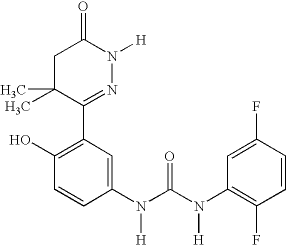

### N-[4-({[(3-Chloro-4-fluorophenyl)amino]carbonyl}amino)phenyl]acetamide

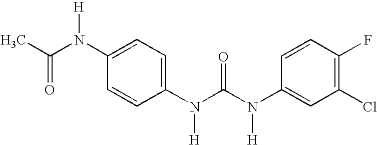

### N-[4-({[(3-Chloro-4-fluorophenyl)amino]carbonyl}amino)phenyl]pentanamide

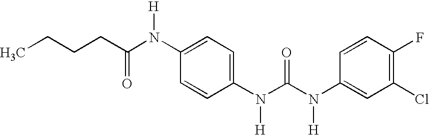

### N-[3-({[(3-Chloro-4-fluorophenyl)amino]carbonyl}amino)phenyl]-1-butanesulfonamide

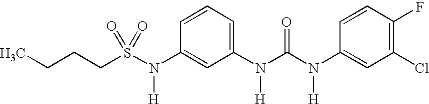

### 1-(3-Chloro-4-fluorophenyl)-3-[3-(4-isopropyl-5-oxo-4,5-dihydro-1H-[1,2,4]triazol-3-yl)phenyl]urea

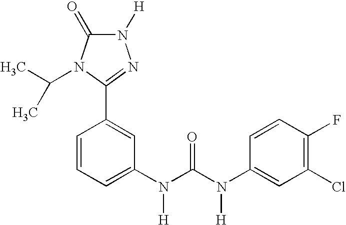

### 1-(3-Chloro-4-fluorophenyl)-3-[3-(4-cyclohexyl-5-oxo-4,5-dihydro-1H-[1,2,4]triazol-3-yl)phenyl]urea

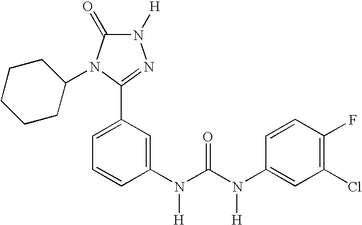

### N-(4-Chloro-2-methylphenyl)-N′-[3-(4,4-dimethyl-5-oxo-4,5-dihydro-1H-pyrazol-3-yl)phenyl]urea

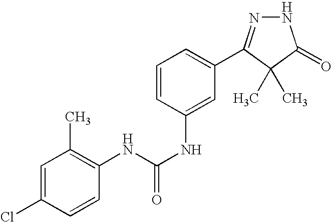

The compounds of the invention may, depending on their structure, exist in stereoisomeric forms (enantiomers, diastereoisomers). The invention therefore relates to the enantiomers or diastereoisomers and respective mixtures thereof. The stereoisomerically pure constituents can be isolated in a known manner from such mixtures of enantiomers and/or diastereoisomers.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Physiologically acceptable salts of the compounds (I) also include salts of usual bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates refer for the purposes of the invention to those forms of the compounds which form a complex in the solid or liquid state by coordination with solvent molecules. Hydrates are a special form of solvates in which the coordination takes place with water.

For the purposes of the present invention, the substituents have the following meaning, unless specified otherwise:

Alkyl per se and “alk” and “alkyl” in alkoxy, alkylamino, alkylaminocarbonyl and alkoxycarbonyl stand for a linear or branched alkyl radical usually having 1 to 6, preferably 1 to 4, particularly preferably 1 to 3, carbon atoms, by way of example and preferably methyl, ethyl, n-propyl, isopropyl, tert-butyl, n-pentyl and n-hexyl.

Alkoxy is by way of example and preferably methoxy, ethoxy, n-propoxy, isopropoxy, tert-butoxy, n-pentoxy and n-hexoxy.

Alkylamino is an alkylamino radical having one or two (chosen independently of one another) alkyl substituents, by way of example and preferably methylamino, ethylamino, n-propylamino, isopropylamino, tert-butylamino, n-pentylamino, n-hexylamino, N,N-dimethylamino, N,N-diethylamino, N-ethyl-N-methyl amino, N-methyl-N-n-propylamino, N-isopropyl-N-n-propylamino, N-tert-butyl-N-methylamino, N-ethyl-N-n-pentylamino and N-n-hexyl-N-methylamino.

Alkylaminocarbonyl is an alkylaminocarbonyl radical having one or two (chosen independently of one another) alkyl substituents, by way of example and preferably methylaminocarbonyl, ethylaminocarbonyl, n-propylaminocarbonyl; isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylaminocarbonyl and N-n-hexyl-N-methylaminocarbonyl.

Alkoxycarbonyl is by way of example and preferably methoxycarbonyl, ethoxycarbonyl, n-propoxycarbonyl, isopropoxycarbonyl, tert-butoxycarbonyl, n-pentoxycarbonyl and n-hexoxycarbonyl.

Cycloalkyl is a cycloalkyl group usually having 3 to 8, preferably 5 to 7, carbon atoms, by way of example and preferably cyclopropyl, cyclobutyl, cyclopentyl, cyclohexyl, cycloheptyl and adamantyl.

Aryl is a mono- to tricyclic aromatic, carbocyclic radical usually having 6 to 14 carbon atoms; by way of example and preferably phenyl, naphthyl and phenanthrenyl.

5- to 10-membered heteroaryl (“C₅-C₁₀-heteroaryl”) stands for the purposes of the invention for 5- to 10-membered aromatic rings which comprise heteroatoms and have at least one aromatic ring, which may comprise 1 to 4 heteroatoms which are selected from O, S and N. Heteroaryl may in turn also be substituted via C or N. Examples which may be mentioned are: pyridyl, furyl, thienyl, pyrrolyl, imidazolyl, pyrazolyl, pyrazinyl, pyrimidinyl, pyridazinyl, indolicenyl, indolyl, benzo[b]thienyl, benzo[b]furyl, indazolyl, quinolyl, isoquinolyl, naphthyridinyl, quinazolinyl, etc.

A 5- to 7-membered saturated or partially unsaturated heterocycle (“C₅-C₇-heterocyclyl”) having up to 3 heteroatoms from the series S, N and/or O is for the purposes of the invention generally a mono- or polycyclic, preferably mono- or bicyclic heterocycle which may comprise one or more double bonds and which is linked via a ring carbon atom or a ring nitrogen atom. Heterocyclyl may in turn also be substituted via C or N. Examples which may be mentioned are: tetrahydrofuryl, pyrrolidin-2-yl, pyrrolidin-3-yl, pyrrolinyl, piperidinyl, dihydropyridinyl, piperazinyl, morpholinyl, azepinyl, diazepinyl. Preference is given to piperidinyl, morpholinyl and pyrrolidinyl.

Halogen is fluorine, chlorine, bromine and iodine, preferably fluorine and chlorine.

The compounds of the formula (Ia) and (Ib) are known or can be prepared by the following methods.

In method

[A] compounds of the general formula (IIa), 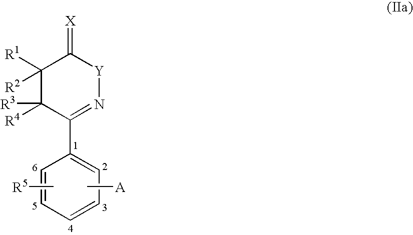
in which
A is linked via positions 2, 3, 5 or 6 to the aromatic system, and
R¹, R², R³, R⁴, R⁵, A, X and Y have the meaning indicated above,
are reacted with compounds of the general formula (IIIa), 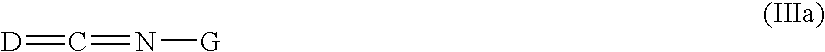
in which
D and G have the meaning indicated above,
to give compounds of the general formula (Iaa), 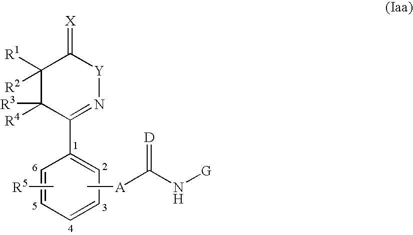
in which
A is linked via positions 2, 3, 5 or 6 to the aromatic system, and
R¹, R², R³, R⁴, R⁵, A, D, G, X and Y have the meaning indicated above,
in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as ethyl acetate, acetone, dimethylformamide, dimethylacetamide, 2-butanone, dimethyl sulfoxide, acetonitrile or pyridine, the preferred solvents being tetrahydrofuran or methylene chloride, where appropriate in the presence of a base such as, for example, alkali metal carbonates such as cesium carbonate, sodium or potassium carbonate, or potassium tert-butoxide, or other bases such as sodium hydride, DBU, triethylamine or diisopropylethylamine, preferably triethylamine, preferably in a temperature range from room temperature to the reflux of the solvents under atmospheric pressure.
The compounds of the general formula (IIa) are prepared below as (IIaa), (IIba) and (IIca).
The compounds of the general formula (IIIa) are known or can be synthesized from the appropriate precursors by known methods.

In method

[B] compounds of the general formula (IIa) are reacted with compounds of the general formula (IVa), 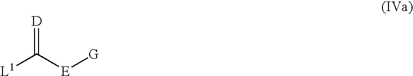
in which
D, E and G have the meaning indicated above, and
L¹ is p-nitrophenyl or halogen, preferably bromine or chlorine, to give compounds of the general formula (Iaa), 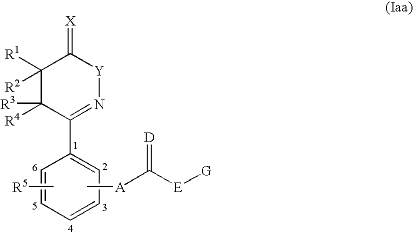
in which
A is linked via positions 2, 3, 5 or 6 to the aromatic system, and
R¹, R², R³, R⁴, R⁵, A, D, E, G, X and Y have the meaning indicated above,
in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as ethyl acetate, acetone, dimethylformamide, dimethylacetamide, 2-butanone, acetonitrile or pyridine, the preferred solvents being tetrahydrofuran or methylene chloride, where appropriate in the presence of a base such as, for example, alkali metal carbonates such as cesium carbonate, sodium or potassium carbonate, or potassium tert-butoxide, or other bases such as sodium hydride, DBU, triethylamine or diisopropylethylamine, preferably triethylamine, preferably in a temperature range from room temperature to the reflux of the solvents under atmospheric pressure.
The compounds of the general formula (IVa) are known or can be synthesized from the appropriate precursors by methods known per se.

In method

[C] compounds of the general formula (Va), 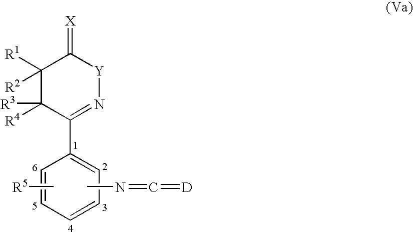
in which
—NCD is linked via positions 2, 3, 5 or 6 to the aromatic system, and
R¹, R², R³, R⁴, R⁵, D, X and Y have the meaning indicated above,
are reacted with compounds of the general formula (VIa),
   H-M-G (VIa)
in which
G has the meaning indicated above, and
M is oxygen or NR⁷,
   in which
   R⁷ has the meaning indicated above,
to give compounds of the general formula (Iba), 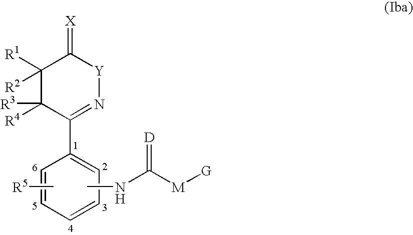
in which
—NH—C(D)-M-G is linked via positions 2, 3, 5 or 6 to the aromatic system, and
R¹, R², R³, R⁴, R⁵, D, G, M, X and Y have the meaning indicated above,
in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as ethyl acetate, acetone, dimethylformamide, dimethylacetamide, 2-butanone, dimethyl sulfoxide, acetonitrile or pyridine, the preferred solvents being tetrahydrofuran or methylene chloride, where appropriate in the presence of a base such as, for example, alkali metal carbonates such as cesium carbonate, sodium or potassium carbonate, or potassium tert-butoxide, or other bases such as sodium hydride, DBU, triethylamine or diisopropylethylamine, preferably triethylamine, preferably in a temperature range from room temperature to the reflux of the solvents under atmospheric pressure.

The compounds of the general formula (VIa) are known or can be synthesized from the appropriate precursors by methods known per se.

Compounds of the general formula (IIaa), 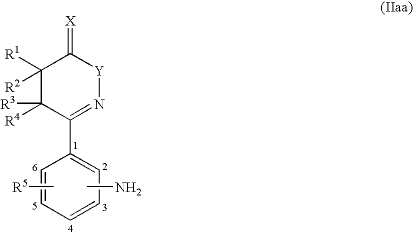
in which
NH₂ is linked via positions 2, 3, 5 or 6 to the aromatic system, and
R¹, R², R³, R⁴, R⁵, X and Y have the meaning indicated above,
   are prepared by initially reacting compounds of the general formula (VIIa), 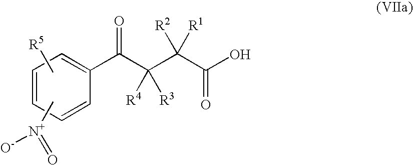
   in which
NO₂ is linked via positions 2, 3, 5 or 6 to the aromatic system, and
R¹, R², R³, R⁴ and R⁵ have the meaning indicated above,
   in the case where X is oxygen,
   with hydrazine, hydroxylamine or a compound of the general formula (VIIIa), 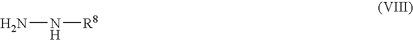
   in which
R⁸ has the meaning indicated above,
   and subsequently reducing the nitro group to the amino group. These two reactions can take place in one or two reaction steps.

In a one-stage method, reaction takes place with hydrazine and with palladium on carbon simultaneously in inert solvents, which include ethers such as diethyl ether, methyl-tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylacetamide, acetonitrile or pyridine, preferred solvents being ethanol or isopropanol, preferably in a temperature range from room temperature to reflux of the solvents under atmospheric pressure.

In a two-stage method, reaction initially takes place with hydrazine, hydroxylamine or a compound of the general formula (VIIIa) in inert solvents, which include ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylformamide, dimethylacetamide, acetonitrile or pyridine, preferred solvents being ethanol or isopropanol, preferably in a temperature range from room temperature to reflux of the solvents under atmospheric pressure.

In the second stage, reaction takes place with hydrogen donors, preferably hydrazine or hydrogen and with palladium on carbon, or with tin dichloride in inert solvents, which include ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as ethyl acetate, dimethylformamide, dimethylacetamide, acetonitrile or pyridine, preferred solvents being ethanol, isopropanol or, in the case of tin dichloride, in dimethylformamide, preferably in a temperature range from room temperature to reflux of the solvents under atmospheric pressure up to 3 bar.

In the case where X is sulfur,

reaction initially takes place with hydrazine, hydroxylamine or a compound of the general formula (VIIIa), and then the oxygen is replaced by sulfur using Lawesson's reagent, and subsequently the nitro group is reduced to the amino group.

In the first stage, reaction takes place initially with hydrazine, hydroxylamine or a compound of the general formula (VIIIa) in inert solvents, which include ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylformamide, dimethylacetamide, acetonitrile or pyridine, preferred solvents being ethanol or isopropanol, preferably in a temperature range from room temperature to reflux of the solvents under atmospheric pressure.

In the second stage, is carried out with Lawesson's reagent in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, 1,2-dimethoxyethane, dimethyl sulfoxide or pyridine, with preference for toluene, xylene or dioxane, preferably in a temperature range from room temperature to reflux of the solvents under atmospheric pressure.

In the third stage, reaction takes place with hydrogen donors, preferably hydrazine or hydrogen and with palladium on carbon, or with tin dichloride in inert solvents, which include ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylformamide, dimethylacetamide, acetonitrile or pyridine, preferred solvents being ethanol, isopropanol or, in the case of tin dichloride, in dimethylformamide, preferably in a temperature range from room temperature to reflux of the solvents under atmospheric pressure up to 3 bar.

Compounds of the general formula (VIIa) can exist in two different forms. Only the open-chain form is drawn when describing the methods.

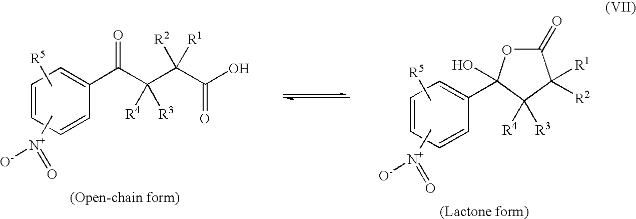

Compounds of the general formula (IIba), 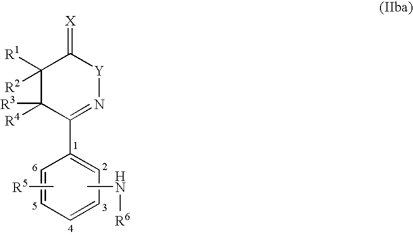
in which
NHR⁶ is linked via positions 2, 3, 5 or 6 to the aromatic system, and
R¹, R², R³, R⁴, R⁵, R⁶, X and Y have the meaning indicated above,
   are prepared by reacting compounds of the general formula (IIaa) with compounds of the general formula (IXa),
   L²-R⁶ (IXa)
   in which
R⁶ has the meaning indicated above, and
L² is halogen, preferably bromine or iodine,
   in inert solvents, which include ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as dimethylformamide, dimethylacetamide, acetonitrile or pyridine, preferred solvents being tetrahydrofuran or diethyl ether, where appropriate in the presence of a base such as, for example, alkali metal hydroxides such as sodium or potassium hydroxide, or alkali metal carbonates such as cesium carbonate, sodium or potassium carbonate, or amides such as sodium amide, lithium bis(trimethylsilyl)amide, lithium diisopropylamide, or organometallic compounds such as butyllithium or phenyllithium, or other bases such as sodium hydride, DBU, triethylamine or diisopropylethylamine, preferably diisopropylethylamine, potassium tert-butoxide or DBU, preferably in a temperature range from room temperature to reflux of the solvents under atmospheric pressure.

The compounds of the general formula (IXa) are known or can be synthesized from the appropriate precursors by known methods.

Compounds of the general formula (IIca), 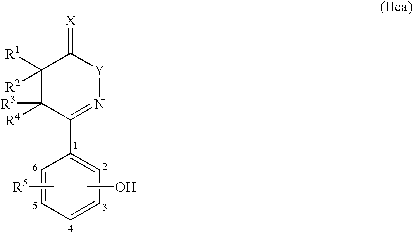
in which
OH is linked via positions 2, 3, 5 or 6 to the aromatic system, and
R¹, R², R³, R⁴, R⁵, X and Y have the meaning indicated above,
   are prepared by initially preparing from compounds of the general formula (IIaa) the diazonium compounds by methods known to the skilled worker, and subsequently boiling them to give the phenols (cf. Organikum, 17th edition, VEB Deutscher Verlag der Wissenschaften, Berlin, page 543).

Compounds of the general formula (Va) are prepared by reacting compounds of the general formula (IIaa)

with trichloromethyl chloroformate

in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as ethyl acetate, acetone, dimethylformamide, dimethylacetamide, 2-butanone, acetonitrile or pyridine. Preferred solvents are tetrahydrofuran or dichloromethane, where appropriate in the presence of a base such as, for example, 1,8-bis(dimethylamino)naphthalene, DBU, triethylamine or diisopropylethylamine, preferably 1,8-bis(dimethylamino)naphthalene, preferably in a temperature range from room temperature to reflux of the solvents under atmospheric pressure.
The compounds of the general formula (VIIa) are prepared by reacting compounds of the general formula (Xa), 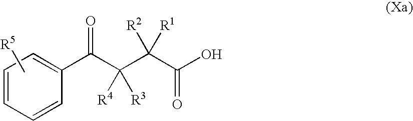
in which
R¹, R², R³, R⁴ and R⁵ have the meaning indicated above,
with fuming nitric acid, concentrated nitric acid or nitration acid preferably in a temperature range from −30° C. to 0° C. under atmospheric pressure.

Compounds of the general formula (Xa) can exist in two different forms. Only the open-chain form is drawn when describing the methods.

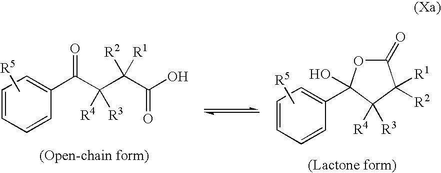

Compounds of the general formula (Xa) are prepared by reacting compounds of the general formula (XIa), 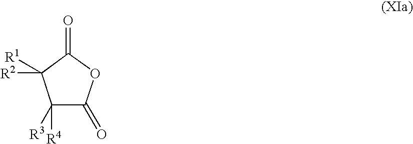
in which
R¹, R², R³ and R⁴ have the meaning indicated above,
   with compounds of the general formula (XIIa), 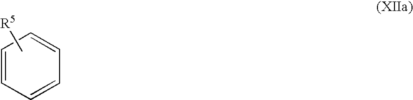
   in which
R⁵ has the meaning indicated above,
   with Lewis acids, preferably aluminum trichloride,
   in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, nitrobenzene, hexane, cyclohexane or petroleum fractions, or other solvents such as ethyl acetate, acetone, dimethylformamide, dimethylacetamide, 2-butanone, dimethyl sulfoxide, acetonitrile or pyridine (1,2-dichloroethane is preferred as solvent) preferably in a temperature range from −20° C. to room temperature under atmospheric pressure.

The compounds of the general formula (XIa) and (XIIa) are known or can be synthesized from the appropriate precursors by known methods.

As an alternative synthetic route for preparing the compounds of the general formula (Xaa), which are compounds of the general formula (Xa) in which
R² is hydrogen,
   compounds of the general formula (XIIIaa), 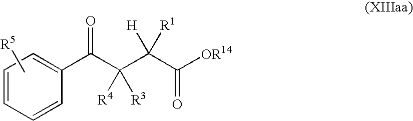
   in which
R¹, R³, R⁴ and R⁵ have the meaning indicated above, and
R¹⁴ is (C₁-C₆)alkyl, preferably methyl and ethyl,
   are reacted with bases such as, for example, alkali metal hydroxides such as sodium, lithium or potassium hydroxide, or alkali metal carbonates such as cesium carbonate, sodium or potassium carbonate, preferably sodium hydroxide, in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylformamide, dimethylacetamide, dimethyl sulfoxide, acetonitrile or pyridine, or mixtures of solvents (tetrahydrofuran and/or methanol are preferred as solvents) preferably in a temperature range from 0° C. to room temperature under atmospheric pressure.

The compounds of the general formula (Xa) can also be prepared in analogy to the synthetic route described for methods of compounds of the general formula (Xaa) from the compounds of the general formula (XIIIa).

Compounds of the general formula (XIIIaa) are prepared by reacting compounds of the general formula (XIVa) 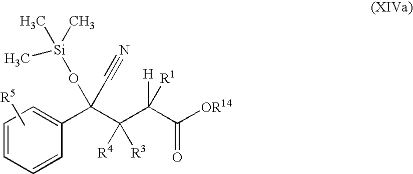
in which
R¹, R³, R⁴, R⁵ and R¹⁴ have the meaning indicated above,
   with tetrabutylammonium fluoride
   in inert solvents, which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as nitromethane, ethyl acetate, acetone, dimethylformamide, dimethylacetamide, 2-butanone, dimethyl sulfoxide, acetonitrile or pyridine (tetrahydrofuran is preferred as solvent) preferably in a temperature range from 0° C. to room temperature under atmospheric pressure.

Compounds of the general formula (XIVa) are prepared by reacting compounds of the general formula (XVa), 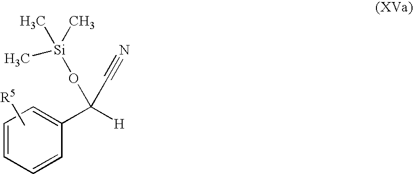
in which
R⁵ has the meaning indicated above,
   with compounds of the general formula (XVIa), 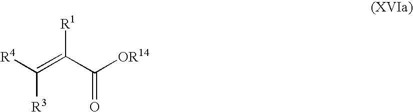
   in which
R¹, R³, R⁴ and R¹⁴ have the meaning indicated above,
   in inert solvents, which include ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, ethylbenzene, xylene, toluene, hexane, heptane, cyclohexane or petroleum fractions, or other solvents such as dimethylformamide, dimethylacetamide, acetonitrile or pyridine, or mixtures of the solvents, preferred solvents being diethyl ether, tetrahydrofuran, heptane and/or ethyl benzene, where appropriate in the presence of a base such as, for example, alkali metal hydroxides such as sodium or potassium hydroxide, or alkali metal carbonates such as cesium carbonate, sodium or potassium carbonate, or sodium or potassium methanolate, or sodium or potassium ethanolate or potassium tert-butoxide, or amides such as sodium amide, lithium bis(trimethylsilyl)amide, lithium diisopropylamide, or organometallic compounds such as butyllithium or phenyllithium, or other bases such as sodium hydride, DBU, triethylamine or diisopropylethylamine, preferably lithium diisopropylamide, preferably in a temperature range from −78° C. to room temperature under atmospheric pressure.

The compounds of the general formula (XVIa) are known or can be synthesized from the appropriate precursors by known methods.

Compounds of the general formula (XVa) are prepared by reacting compounds of the general formula (XVIIa) 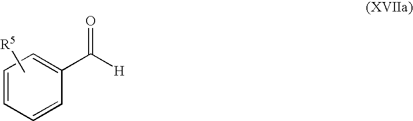
in which
R⁵ has the meaning indicated above,
   with trimethylsilyl cyanide and zinc iodide
   where appropriate in inert solvents which include halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as nitromethane, ethyl acetate, acetone, dimethylformamide, dimethylacetamide, 2-butanone, dimethyl sulfoxide, acetonitrile or pyridine (tetrahydrofuran is preferred as solvent) preferably in a temperature range from room temperature to 100° C. under atmospheric pressure.

The compounds of the general formula (XVIIa) are known or can be synthesized from the appropriate precursors by known methods.

Compounds of the general formula (XIIIa), 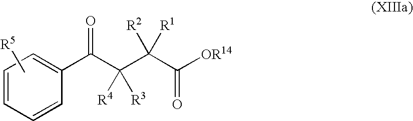
in which
R¹, R², R³, R⁴, R⁵ and R¹⁴ have the meaning indicated above,
   are prepared by reacting compounds of the general formula (XVIIIa), 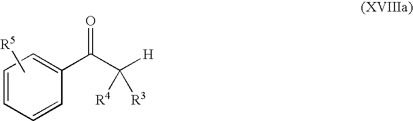
   in which
R³, R⁴ and R⁵ have the meaning indicated above,
   with compounds of the general formula (XIXa), 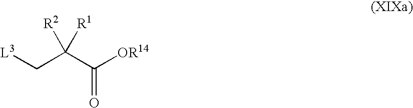
   in which
R¹, R² and R¹⁴ have the meaning indicated above, and
L³ is halogen, preferably bromine or iodine,
   in inert solvents, which include ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, ethylbenzene, xylene, toluene, preferred solvents being tetrahydrofuran or toluene, where appropriate in the presence of a base such as, for example, amides such as sodium amide, lithium hexamethyldisilazide, potassium hexamethyldisilazide, lithium diisopropylamide, or other bases such as sodium hydride, DBU or diisopropylethylamine, preferably sodium amide, lithium hexamethyldisilazide, potassium hexamethyldisilazide or lithium diisopropylamide, preferably in a temperature range from −78° C. to room temperature under atmospheric pressure.

The compounds of the general formula (XVIIIa) and (XIXa) are known or can be synthesized from the appropriate precursors by known methods (for (XVIIIa) compare M. R. Schneider, H. Ball, J. Med. Chem. 1986, 29, 75-79; Robl, et al., Synthesis 1991, 56; J. Org. Chem. 1996, 61, 607).

In an alternative synthetic route for preparing compounds of the general formula (IIaaa), which are compounds of the general formula (IIaa) in which
R¹ and R² are hydrogen,
   compounds of the general formula (XXa), 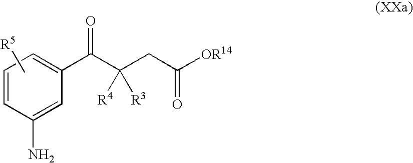
   in which
R³, R⁴, R⁵ and R¹⁴ have the meaning indicated above,
   are reacted with hydrazine. The reaction takes place in analogy to the first stage of the two-stage method which is described for preparing compounds of the general formula (IIaa).

The compounds of the general formula (XXa) are prepared by reacting compounds of the general formula (XXIa), 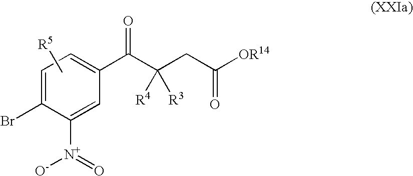
in which
R³, R⁴, R⁵ and R¹⁴ have the meaning indicated above,
   with reducing agents. The reaction takes place in analogy to the second stage of the two-stage method which is described for preparing the compounds of the general formula (IIaa).

The compounds of the general formula (XXIa) are prepared by reacting compounds of the general formula (XXIIa), 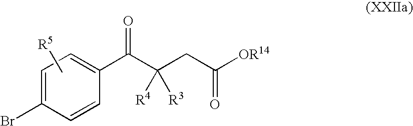
in which
R³, R⁴, R⁵ and R¹⁴ have the meaning indicated above,
   with fuming nitric acid, concentrated nitric acid or nitration acid in analogy to the method which is described for preparing the compounds of the general formula (VIIa).

The compounds of the general formula (XXIIa) can be synthesized from the appropriate precursors by the method described for the compounds of the general formula (XIIIa).

In an alternative synthetic route for preparing the compounds of the general formula (XXa), compounds of the general formula (XXIIIa), 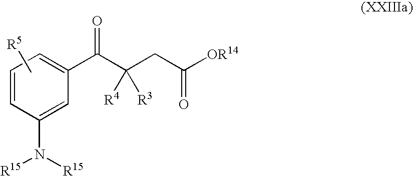
in which
R³, R⁴, R⁵ and R¹⁴ have the meaning indicated above,
and
R¹⁵ is allyl or benzyl,
   are reacted in the case of benzayl with reducing agents. The reaction takes place in analogy to the second stage of the two-stage method which is described for preparing the compounds of the general formula (IIaa).

In the case of allyl, a method with tetrakistriphenylphosphinepalladium and N,N-dimethylbarbituric acid is used, compare F. Garro-Helion, A. Merzouk, F. Guibe, J. Org. Chem. 1993, 58, 6109-6113.

The compounds of the general formula (XXIIIa) can be synthesized from the appropriate precursors by the method described for the compounds of the general formula (XIIIa).

The methods described above can be illustrated by way of example by the following formula diagrams:

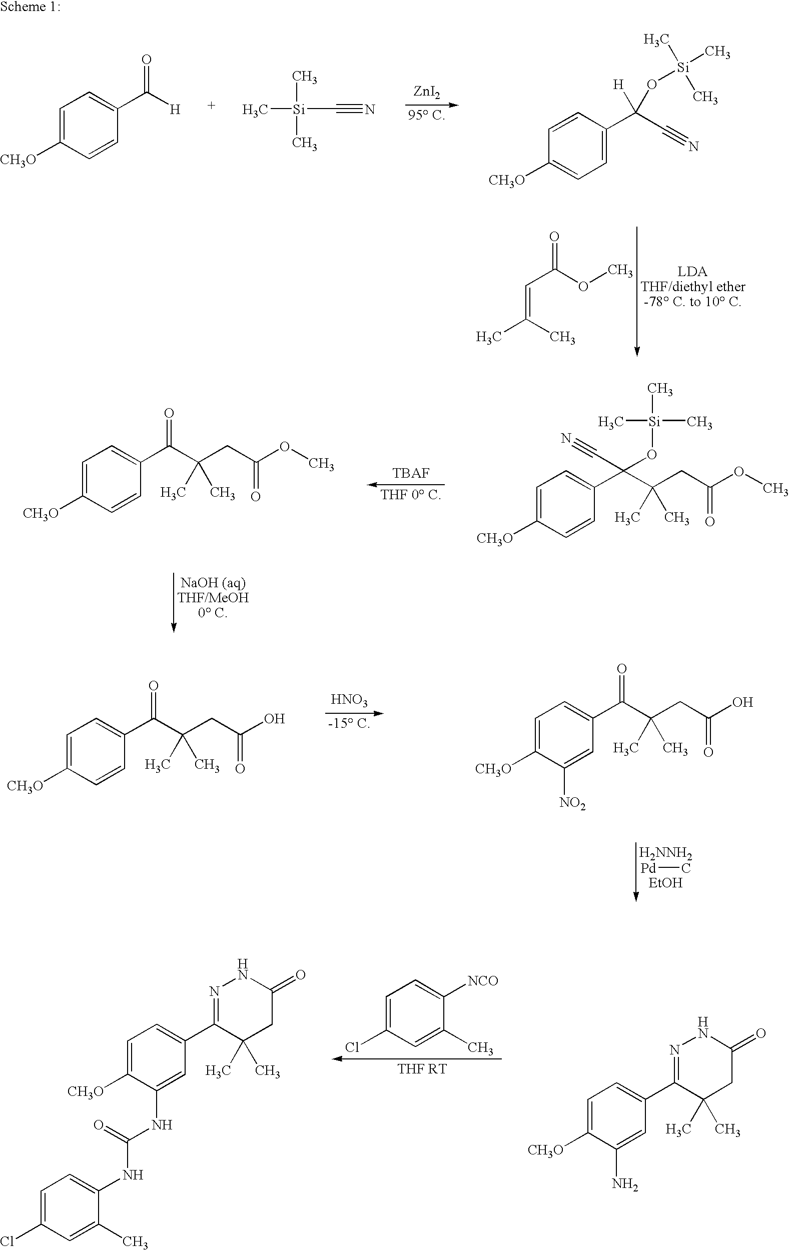

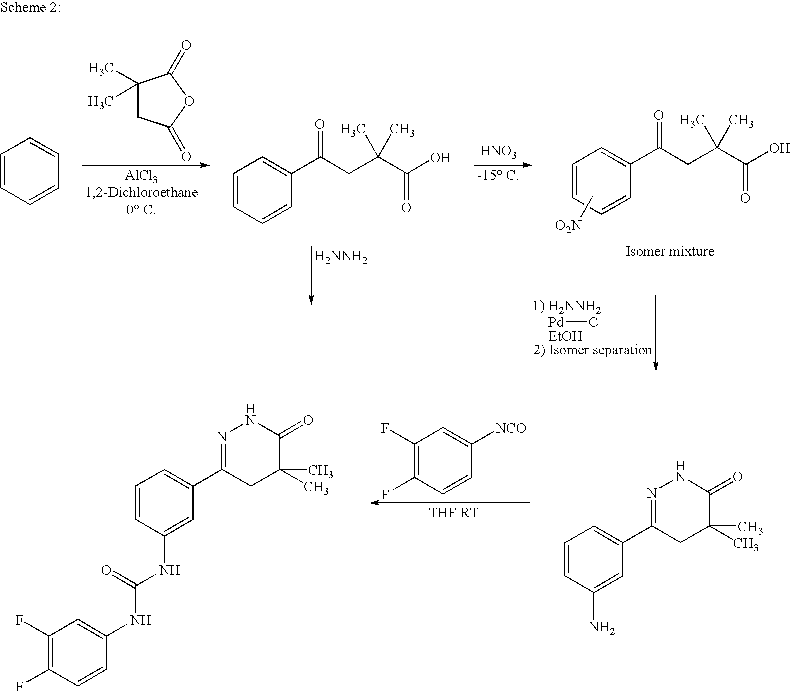

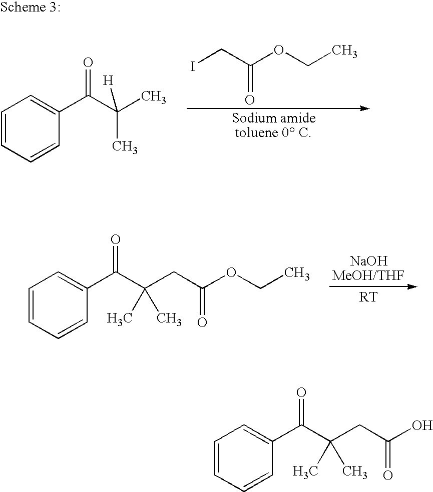

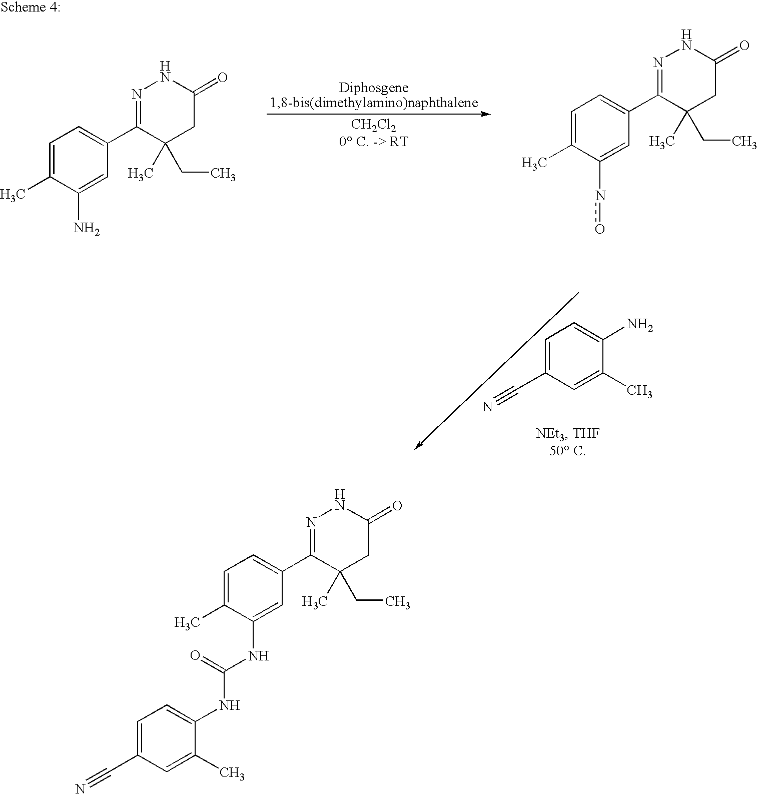

In method

[D] compounds of the general formula (IIb), 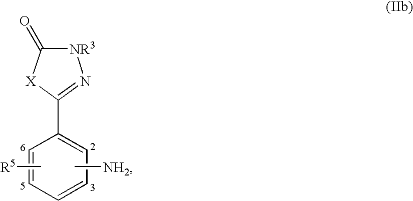
in which
NH₂ is linked via one of positions 2, 3, 5 or 6 to the aromatic system, and
X, R³ and R⁵ have the meaning indicated above,
   are reacted with compounds of the formula (IIIb)
   DCN—R² (IIIb),
   in which R² and D have the meaning indicated above.

The reaction takes place in inert solvents, where appropriate in the presence of a base, preferably in a temperature range from room temperature to reflux of the solvents under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as ethyl acetate, acetone, dimethylformamide, dimethylacetamide, 2-butanone, dimethyl sulfoxide, acetonitrile or pyridine, with preference for tetrahydrofuran or methylene chloride.

Examples of bases are alkali metal carbonates such as cesium carbonate, sodium or potassium carbonate, or potassium tert-butoxide, or other bases such as sodium hydride, DBU, triethylamine or diisopropylethylamine, with preference for diisopropylethylamine and triethylamine.

The compounds of the formula (IIIb) are known or can be synthesized from the appropriate precursors by known methods.

The compounds of the formula (IIab), which are compounds of the formula (IIb) in which X is 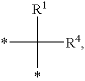
can be prepared by reducing compounds of the formula (IVb) 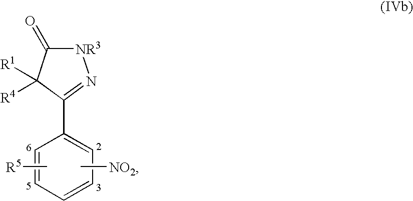
in which
NO₂ is linked via one of positions 2, 3, 5 or 6 to the aromatic system, and
R¹, R³, R⁴ and R⁵ have the meaning indicated above
   for example with tin(II) chloride or hydrogen with palladium on carbon.

The reaction takes place in inert solvents, preferably in a temperature range from room temperature to reflux of the solvents under atmospheric pressure up to 3 bar.

Examples of inert solvents are ethers such as diethyl ether, diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylformamide, dimethylacetamide, acetonitrile or pyridine, with preference for ethanol, isopropanol or, in the case of tin dichloride, in dimethylformamide.

The compounds of the formula (IVb) can be prepared by reacting compounds of the formula (Vb) 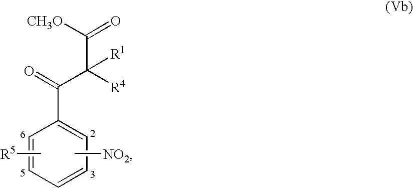
in which
NO₂ is linked via one of positions 2, 3, 5 or 6 to the aromatic system, and
R¹, R⁴ and R⁵ have the meaning indicated above,
   with hydrazine or a compound of the general formula (VIb), 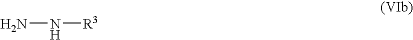
   in which
R³ has the meaning indicated above.

The reaction takes place in inert solvents, preferably in a temperature range from room temperature to reflux of the solvents under atmospheric pressure.

Examples of inert solvents are ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as dimethylformamide, dimethylacetamide, acetonitrile or pyridine, with preference for ethanol or isopropanol.

The compounds of the formula (VIb) are known or can be synthesized from the appropriate precursors by known methods.

The compounds of the formula (Vb) can be prepared by reacting compounds of the formula (VIIb) 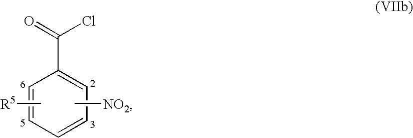
in which
NO₂ is linked via one of positions 2, 3, 5 or 6 to the aromatic system, and
R⁵ has the meaning indicated above,
   with compounds of the formula (VIIIb) 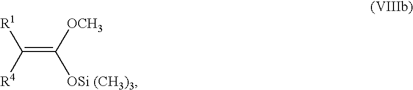
   in which R¹ and R⁴ have the meaning indicated above,
   in the presence of boron trifluoroetherate.

The reaction takes place in inert solvents, preferably in a temperature range from room temperature to reflux of the solvents under atmospheric pressure.

Examples of inert solvents are ethers such as diethyl ether, methyl tert-butyl ether, 1,2-dimethoxyethane, dioxane, tetrahydrofuran, glycol dimethyl ether or diethylene glycol dimethyl ether, hydrocarbons such as benzene, xylene, toluene, hexane, cyclohexane or petroleum fractions, or other solvents such as dimethylacetamide, acetonitrile or pyridine, with preference for diethyl ether.

The compounds of the formula (VIIb) are known or can be prepared in analogy to known methods.

The compounds of the formula (VIIIb) are known or can be prepared in analogy to C. Ainsworth, F. Chen, Y.-N. Kuo, J. Organomet. Chem. 1972, 46, 59-71.

The compounds of the formula (IIbb), which are compounds of the formula (IIb), in which X is NR⁶, can be prepared by reacting compounds of the formula (IXb) 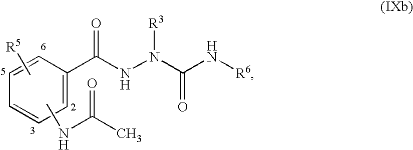
in which
NHC(O)CH₃ is linked via one of positions 2, 3, 5 or 6 to the aromatic system, and
R³, R⁵ and R⁶ have the meaning indicated above,
   in water in the presence of a base, preferably at 60° C. up to reflux of the water under atmospheric pressure.

Examples of bases are alkali metal hydroxides such as sodium, lithium or potassium hydroxide, alkali metal carbonates such as cesium carbonate, sodium or potassium carbonate, with preference for sodium hydroxide.

The compounds of the formula (IXb) can be prepared by reacting compounds of the formula (Xb) 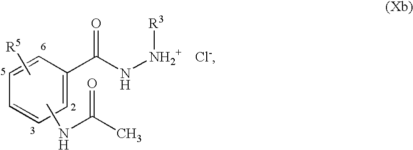
in which
NHC(O)CH₃ is linked via one of positions 2, 3, 5 or 6 to the aromatic system, and
R³ and R⁵ have the meaning indicated above,
   with compounds of the formula (XIb)
   OCN—R⁶ (XIb)
   in which R⁶ has the meaning indicated above,
   by the method described for preparing the compounds of the formula (Ib).

The compounds of the formula (XIb) are known or can be prepared in analogy to known methods.

The compounds of the formula (Xb) can be prepared be reacting compounds of the formula (XIIb) 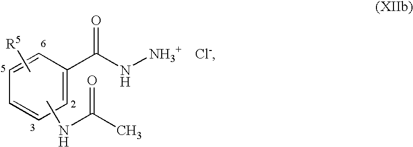
in which
NHC(O)CH₃ is linked via one of positions 2, 3, 5 or 6 to the aromatic system, and
R⁵ has the meaning indicated above,
   with compounds of the formula (XIIIb)
   OHC—R³ (XIIIb)
   in which R³ has the meaning indicated above,
   in a reductive amination by methods known to the skilled worker for reductive aminations.

The compounds of the formulae (XIIb) and (XIIIb) are known or can be prepared in analogy to known methods.

The preparation of the compounds of the invention can be illustrated by the following synthesis schemes 5-12.
Synthesis Schemes:

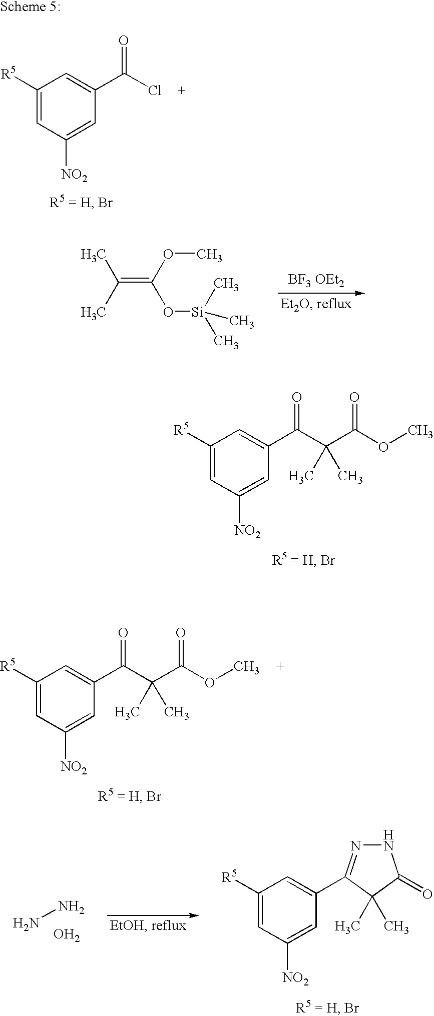

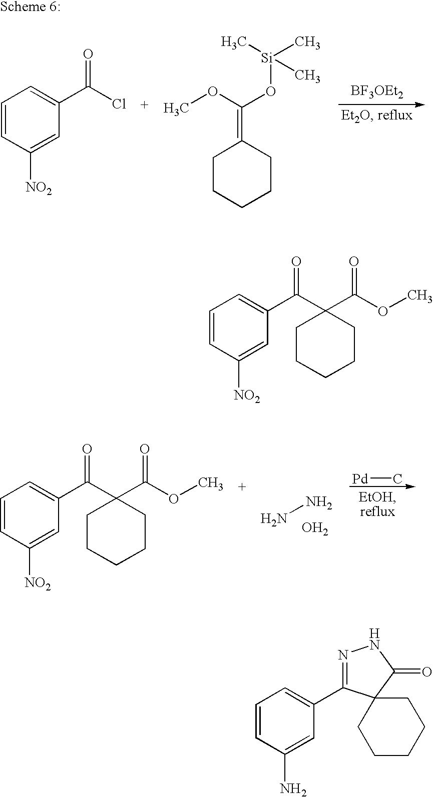

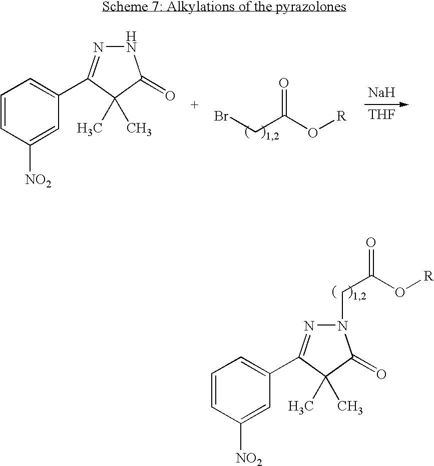

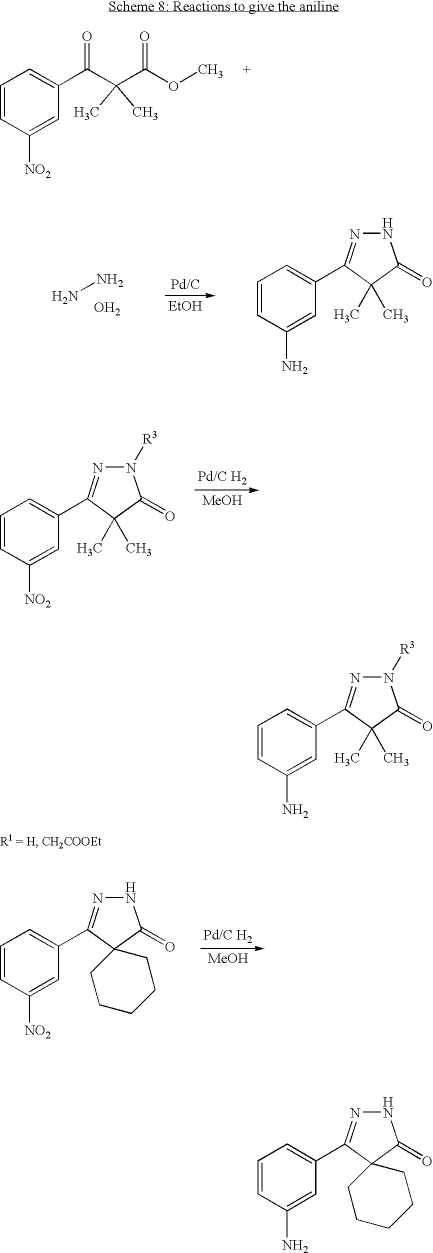

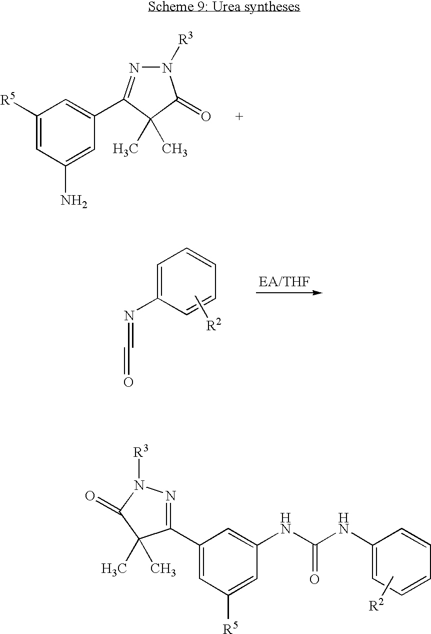

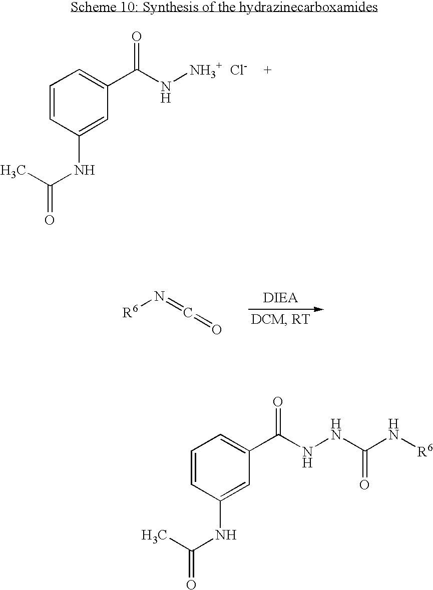

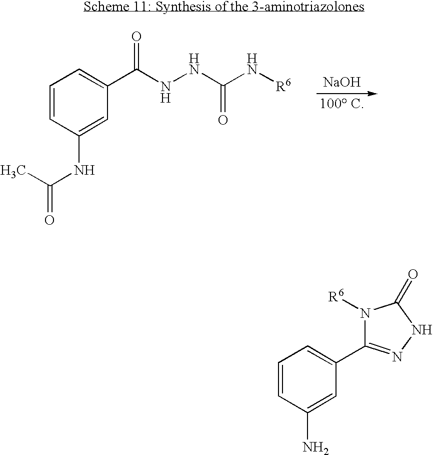

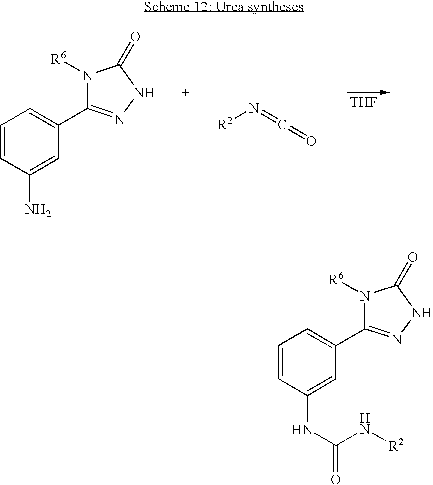

Further compounds of the invention which act by the mechanism disclosed herein are described in the international patent applications WO 03/097595 and WO 03/089421.

The compounds of the invention show a valuable range of pharmacological and pharmacokinetic effects which could not have been predicted.

They are therefore suitable for use as medicaments for the treatment and/or prophylaxis of diseases in humans and animals.

They are notable as UL86 inhibitors.

The compounds of the invention can, because of their pharmacological properties, be employed alone or in combination with other agents for the treatment and/or prevention of herpes infections, in particular infections with human cytomegalovirus (HCMV).

The present invention further relates to medicaments which comprise at least one compound of the invention, preferably together with one or more pharmacologically acceptable excipients or carriers, and to the use thereof for the aforementioned purposes.

The agent may have systemic and/or local effects. It can for this purpose be administered in a suitable way, such as, for example, by the oral, parenteral, pulmonary, nasal, sublingual, lingual, buccal, rectal, transdermal, conjunctival, or otic route or as implant.

For these administration routes it is possible to administer the agent in suitable administration forms.

Suitable for oral administration are administration forms which deliver the agent rapidly and/or in modified fashion, such as, for example, tablets (uncoated and coated tablets, e.g. tablets provided with coatings which are resistant to gastric juice, or film-coated tablets), capsules, sugar-coated tablets, granules, pellets, powders, emulsions, suspensions, solutions and aerosols.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates and sterile powders.

Examples suitable for the other administration routes are pharmaceutical forms for inhalation (inter alia powder inhalers, nebulizers), nasal drops/solutions, sprays; tablets or capsules to be administered lingually, sublingually or buccally, suppositories, preparations for the eyes and ears, vaginal capsules, aqueous suspensions (lotions, shaking mixtures), lipophilic suspensions, ointments, creams, milk, pastes, dusting powders or implants.

The agents can be converted in a manner known per se into the stated administration forms. This takes place with use of inert, non-toxic, pharmaceutically suitable excipients. These include, inter alia, carriers (e.g. microcrystalline cellulose), solvents (e.g. liquid polyethylene glycols), emulsifiers (e.g. sodium dodecyl sulfate), dispersants (e.g. polyvinylpyrrolidone), synthetic and natural biopolymers (e.g. albumin), stabilizers (e.g. antioxidants such as ascorbic acid), colors (e.g. inorganic pigments such as iron oxides) or taste and/or odor-masking agents.

It has generally proved advantageous to administer on parenteral administration amounts of about 0.001 to 10 mg/kg, preferably about 0.01 to 5 mg/kg, of body weight to achieve effective results. On oral administration, the amount is about 0.01 to 25 mg/kg, preferably about 0.1 to 10 mg/kg, of body weight.

It may nevertheless be necessary where appropriate to deviate from the amounts mentioned, specifically as a function of the body weight, administration route, individual response to the agent, type of preparation and time or interval over which administration takes place. Thus, it may be sufficient in some cases to make do with less than the aforementioned minimum amounts, whereas in other cases the upper limit mentioned must be exceeded. It may in the event of administration of larger amounts be advisable to divide these into a plurality of individual doses over the day.

The percentage data in the following tests and examples are percentages by weight unless indicated otherwise; parts are parts by weight. Solvent ratios, dilution ratios and concentration data for liquid/liquid solutions are based in each case on volume.

### A. EXAMPLES

Boc Butoxycarbonyl

DCI Direct chemical ionization (in MS)

DCM Dichloromethane

DIEA N,N-Diisopropylethylamine

DMSO Dimethyl sulfoxide

DMF Dimethylformamide

EA Ethyl acetate (acetic acid ethyl ester)

ESI Electrospray ionization (in MS)

h Hour

HPLC High pressure, high performance liquid chromatography

LC-MS Coupled liquid chromatography-mass spectroscopy

m.p. Melting point

MS Mass spectroscopy

moi Multiplicity of infection

NMR Nuclear magnetic resonance spectroscopy

RP-HPLC Reverse phase HPLC

RT Room temperature

R_{f} Retention index (in TLC)

Rₜ Retention time (in HPLC)

SI Selectivity index

THF Tetrahydrofuran

TLC Thin layer chromatography

HPLC and LCMS Methods:

Method 1 (LCMS): Instrument: Micromass Quattro LCZ, HP100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C.; flow rate: 0.5 ml/min; UV detection: 208-400 nm.

Method 2 (HPLC): Instrument: Finnigan MAT 900S, TSP: P4000, AS3000, UV3000HR; column: Symmetry C 18, 150 mm×2.1 mm, 5.0 μm; eluent C: water, eluent B: water+0.3 g of 35% strength hydrochloric acid, eluent A: acetonitrile; gradient: 0.0 min 2% A→2.5 min 95% A→5 min 95% A; oven: 70° C.; flow rate: 1.2 ml/min; UV detection: 210 nm.

Method 3: Column: Kromasil C18 60*2, L-R temperature: 30° C., flow rate=0.75 mlmin⁻¹, eluent: A=0.005 M HClO₄, B=acetonitrile, gradient:→0.5 min 98% A→4.5 min 10% A→6.5 min 10% A

Starting Compounds

General Procedure [A]:

Synthesis of TMS Cyanohydrins

A spatula tip of anhydrous zinc iodide is added to 55 mmol of trimethylsilyl cyanide in a heat-dried 100 ml three-neck flask under an argon atmosphere. At RT, 50 mmol of the liquid aldehydes are slowly (exothermic reaction) added dropwise (solid aldehydes are added as solid in portions at 60° C.). The resulting brown reaction mixture is heated at 95° C. for 7-8 hours. The product is then distilled with the aid of a Kugelrohr oven under high vacuum. The colorless or slightly yellow liquids obtained thereby are used without further purification for the next reactions.

The following compound is prepared by this procedure:

### Example 1A

### Phenyl[(trimethylsilyl)oxy]acetonitrile

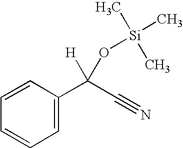

8.80 g (86% of theory) of product are obtained starting from 5.63 g (55 mmol) of trimethylsilyl cyanide with 5.31 g (50 mmol) of benzaldehyde.

HPLC (method 3): Rₜ=3.38 min

MS (DCI): m/z=223 (M+NH₄)⁺

General Procedure [B]:

Reaction of TMS Cyanohydrins with Methyl 3-methyl-2-butenoate

1 eq. of the appropriate TMS cyanohydrin is dissolved in absolute diethyl ether in a heat-dried 250 ml three-neck flask under argon, and the resulting solution is cooled to −78° C. 1.05 eq. of 2 M LDA solution in THF/heptane/ethylbenzene are added dropwise thereto over the course of 30 min. After stirring at this temperature for 30 min, 1 eq. of methyl 3-methyl-2-butenoate, dissolved in a little absolute diethyl ether, is added dropwise. The mixture is allowed to warm to 0° C. to 10° C. over the course of 5 hours. Then saturated ammonium chloride solution is added and stirred for 10 min. The phases are separated and the ethereal phase is washed 2× with saturated ammonium chloride solution. After drying over magnesium sulfate and filtration, the solvent is removed in a rotary evaporator, and the product is obtained and is employed without further purification for the next synthesis step.

The following compound is prepared by this procedure:

### Example 2A

### Methyl 4-cyano-3,3-dimethyl-4-phenyl-4-[(trimethylsilyl)oxy]butanoate

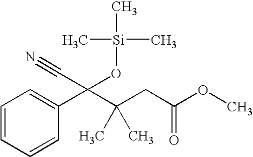

13.69 g (67% of theory) of the title compound are obtained as crude product starting from 8.80 g (43 mmol) of phenyl[(trimethylsilyl)oxy]acetonitrile, after deprotonation with 22.5 ml of 2 M LDA solution, with 5.04 g (43 mmol) of methyl 3-methyl-2-butenoate.

HPLC (method 3): Rₜ=5.53 min

MS (DCI): m/z=337 (M+NH₄)⁺

General Procedure [C]:

Desilylation Using TBAF

1 eq. of the methyl butanoate derivatives is dissolved in absolute THF (0.25 M) under an argon atmosphere and cooled to 0° C. At this temperature, 1.1 eq. of a 1 M TBAF solution in THF are slowly added dropwise. The mixture is stirred for a further 3 hours and then, after addition of water, extracted 3× with dichloromethane. Drying over magnesium sulfate, filtration and removal of the solvent are followed by purification by column chromatography (silica gel: mobile phase cyclohexane/ethyl acetate=85:15) or by Kugelrohr distillation.

The following compound is prepared by this procedure:

### Example 3A

### Methyl 3,3-dimethyl-4-oxo-4-phenylbutanoate

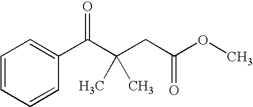

6.54 g (62% of theory) of the title compound are obtained as crude product starting from 13.44 g (42 mmol) of methyl 4-cyano-3,3-dimethyl-4-phenyl-4-[(trimethylsilyl)oxy]-butanoate with 46.3 ml (46.3 mmol) of a 1 M TBAF solution.

HPLC (Method 3): Rₜ=4.25 min

MS (DCI): m/z=238 (M+NH₄)⁺

Alternative Synthesis Method:

48.4 ml (24.20 mmol; 0.5 M solution in toluene) of potassium hexamethyldisilazide are dissolved in 30 ml of tetrahydrofuran and, at −78° C., 3.26 g (22 mmol) of isobutyrophenone in 10 ml of tetrahydrofuran are added. After 2 hours, 4.04 g (26.40 mmol) of methyl bromoacetate are added. After a further 2 hours, 50 ml of 1N hydrochloric acid are added. This is followed by extraction with ethyl acetate. The combined organic phases are washed with water and saturated sodium chloride solution and dried with magnesium sulfate, and the solvent is removed. Preparative normal phase HPLC (column: silica gel, flow rate: 150 ml/min, eluent: isohexane/ethyl acetate=9:1) results in the target compound in a yield of 26%.

HPLC (method 3) Rₜ=4.60 min

MS (DCI/NH₃): m/z=238 (M+NH₄)⁺

General Procedure [D]:

Ester Hydrolysis The ester to be hydrolyzed is dissolved in a THF/methanol mixture (1:1), and the solution is cooled to 0° C. At this temperature, 2 eq. of 1N sodium hydroxide solution are slowly added dropwise. After the reaction has ended (reaction monitored by TLC), equal portions in each case of a 1N sodium hydroxide solution and dichloromethane are added. The organic phase is extracted twice with 1N sodium hydroxide solution. The combined aqueous phases are then acidified with concentrated hydrochloric acid, and the product is extracted three times with dichloromethane. Drying over sodium sulfate, filtration and evaporation of the solvent result in the product, which is used without further purification for the next synthesis step.

The following compound is prepared by this procedure:

### Example 4A

### 5-Hydroxy-4,4-dimethyl-5-phenyldihydro-2(3H)-furanone

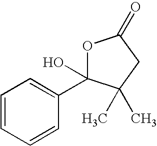

5.20 g (83% of theory) of product are obtained starting from 6.52 g (29.6 mmol) of methyl 3,3-dimethyl-4-oxo-4-phenylbutanoate.

HPLC (method 3): Rₜ=3.88 min

MS (DCI): m/z=224 (M+NH₄)⁺

### Example 5A

### 5-Hydroxy-4,4-dimethyl-5-(3-nitrophenyl)dihydro-2(3H)-furanone and 5-hydroxy-4,4-dimethyl-5-(4-nitrophenyl)dihydro-2(3H)-furanone

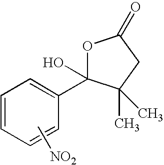

Fuming nitric acid (12 ml) is cooled in a flask under argon to −15° C. At this temperature, 5 g (24.5 mmol) of 5-hydroxy-4,4-dimethyl-5-phenyldihydro-2(3H)-furanone are added as solid in portions. The mixture is stirred at −15° C. for half an hour and then poured into ice and extracted three times with dichloromethane. The combined extracts are dried over magnesium sulfate. Purification takes place by column chromatography (dichloromethane/methanol 97:3). 6.23 g of a product mixture of the title compounds are obtained as crude product.

HPLC (method 3): Rₜ=4.06 min

MS (DCI): m/z=269 (M+NH₄)⁺

### Example 6A

### 6-(3-Aminophenyl)-5,5-dimethyl-4,5-dihydro-3(2H)-pyridazinone and 6-(4-aminophenyl)-5,5-dimethyl-4,5-dihydro-3(2H)-pyridazinone

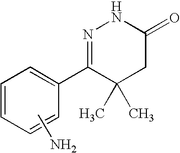

2.98 g (11.9 mmol) of a mixture of 5-hydroxy-4,4-dimethyl-5-(3-nitrophenyl)dihydro-2(3H)-furanone and 5-hydroxy-4,4-dimethyl-5-(4-nitrophenyl)dihydro-2(3H)-furanone are dissolved in 40 ml of ethanol at RT, and 8.91 g (178 mmol) of hydrazine monohydrate are added. Then 300 mg of palladium/carbon (10% by weight) are added, and the reaction mixture is heated to reflux for 20 hours. It is then filtered while hot through Celite, washing with hot ethanol, and evaporated to dryness. The product is crystallized from ethanol. 1.09 g (34% of theory) of a product mixture with 80% of meta and 20% of para product are obtained. Renewed crystallization from the mother liquor affords 1.03 g (30% of theory) of a product mixture with 74% of para and 26% of meta product. The two fractions are combined and separated into the para product and meta product in a preparative HPLC (method 12).

HPLC (method 3): Rₜ=2.53 min (para), and 2.83 min (meta)

MS (EI): m/z=217 (M)⁺

### Example 7A

### 4-(2-Cyano-5-nitrophenyl)-3,3-dimethyl-4-oxobutanoic acid

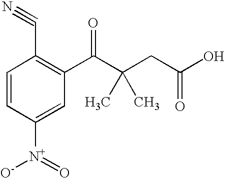

4-(2-Cyano-5-nitrophenyl)-3,3-dimethyl-4-oxobutanoic acid is prepared from 4-(2-fluoro-5-nitrophenyl)-3,3-dimethyl-4-oxobutanoic acid by a method based on the literature Heterocycles 1987, 26, 1227 and Synth. Commun. 1985, 15, 479.

### Example 8A

### 6-(2-Hydroxy-5-nitrophenyl)-5,5-dimethyl-4,5-dihydro-3(2H)-pyridazinone

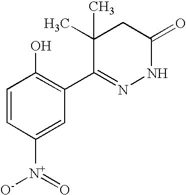

26.00 g (94.12 mmol) of 4-(2-cyano-5-nitrophenyl)-3,3-dimethyl-4-oxobutanoic acid are dissolved in 400 ml of ethanol and, while refluxing, 47.12 g (941.19 mmol) of hydrazine hydrate are added dropwise. The mixture is stirred while boiling for 5 h and then the solution is concentrated to 100 ml. The residue is mixed with water and the volume is concentrated to 200 ml. The crystals are then filtered off with suction and washed with water and diethyl ether. Drying in vacuo results in 20.03 g (81% of theory) of product.

HPLC (method 3): Rₜ=3.50 min

MS (DCI/NH₃): m/z=281 (M+NH₄)⁺.

### Example 9A

### 6-(5-Amino-2-hydroxyphenyl)-5,5-dimethyl-4,5-dihydro-3(2H)-pyridazinone

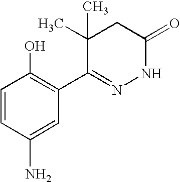

3.00 g (11.40 mmol) of 6-(2-hydroxy-5-nitrophenyl)-5,5-dimethyl-4,5-dihydro-3(2H)-pyridazinone are dissolved in 150 ml of ethanol, and 0.30 g of palladium/carbon (10%) is added. While boiling, 5.70 g (113.96 mmol) of hydrazine hydrate are added dropwise. After stirring under reflux for 18 h, the solvent is removed and the oily residue is crystallized from diethyl ether. It is stirred with water and the crystals are filtered off with suction. Washing with diethyl ether is followed by drying in vacuo. 1.84 g (69% of theory) of product are obtained.

HPLC (method 3): Rₜ=2.30 min

MS (ESI pos): m/z=234 (M+H)⁺.

### Example 10A

### tert-Butyl 4-({[(3-chloro-4-fluorophenyl)amino]carbonyl}amino)phenylcarbamate

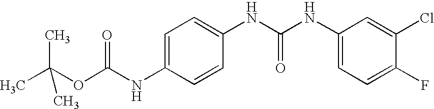

2.34 g (13.61 mmol) of 3-chloro-4-fluorophenyl isocyanate are added to an argon-blanketed suspension of 2.70 g (12.96 mmol) of 4-(tert-butoxycarbonylamino)aniline in 50 ml of dichloromethane at RT. A precipitate immediately separates out. A further 20 ml of dichloromethane are subsequently added.

After a stirring time of 30 min (TLC1 cyclohexane/ethyl acetate 1:1), the precipitate is filtered off with suction and washed with pentane (TLC2 cyclohexane/ethyl acetate 1:1).

The product is predried in a rotary evaporator at 50° C. before being dried under high vacuum.

4.5 g (90% of theory) of product are obtained.

R_{f} (cyclohexane/ethyl acetate 1:1): 0.42

HPLC (method 1): Rₜ=4.43 min

### Example 11A

### N-(4-Aminophenyl)-N′-(3-chloro-4-fluorophenyl)urea

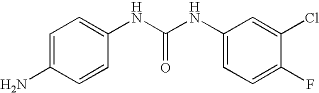

A suspension of 4.53 g (11.93 mmol) of example 10A in 50 ml of 4N hydrogen chloride/dioxane is stirred at RT under argon for 2 h (TLC1 dichloromethane/methanol 100:5). The resulting precipitate is filtered off with suction and washed with dioxane and diethyl ether (TLC2 dichloromethane/methanol/ammonia 9:1:0.1). The product is predried at 50° C. before being dried under high vacuum.

3.5 g (quant.) of product are obtained.

R_{f} (dichloromethane/methanol/ammonia 9:1:0.1): 0.59

HPLC (method 1): Rₜ=2.56 min

### Example 12A

### tert-Butyl 3-({[(3-chloro-4-fluorophenyl)amino]carbonyl}amino)phenylcarbamate

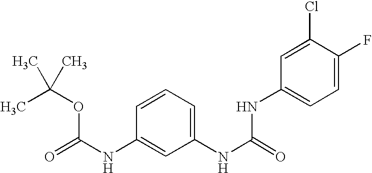

2.59 g (15.13 mmol) of 3-chloro-4-fluorophenyl isocyanate are added to a solution of 3.00 g (14.41 mmol) of 3-(tert-butoxycarbonylamino)aniline in 50 ml of dichloromethane. A precipitate separates out after a few minutes. It is then stirred at RT for 2 h (TLC1 cyclohexane/ethyl acetate 1:1).

The precipitate is filtered off with suction and washed with dichloromethane and diisopropyl ether (TLC2 cyclohexane/ethyl acetate 1:1) and dried under high vacuum.

R_{f} (cyclohexane/ethyl acetate 1:1): 0.63

HPLC (method 2): Rₜ=2.95 min

### Example 13A

### N-(3-Aminophenyl)-N′-(3-chloro-4-fluorophenyl)urea

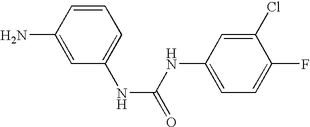

A suspension of 5.00 g (13.16 mmol) of example 12A in 100 ml of 4N hydrogen chloride/dioxane is stirred at RT for 17 h (TLC1 dichloromethane/methanol 100:5). The solid is filtered off with suction, washed with dioxane and diethyl ether (TLC2 dichloromethane/methanol 100:5) and dried under high vacuum for 2 days.

4.6 g (quant.) of product are obtained.

R_{f} (dichloromethane/methanol 100:5): 0.14

HPLC (method 1): Rₜ=3.01 min

General Procedure [E]:

Synthesis of β-keto esters (in analogy to the procedure of M. H. Stefaniak, F. Tinardon, J. D. Wallis, Synlett 1997, 677-678).

1 equivalent of the appropriately substituted 3-nitrobenzoyl chloride is dissolved in absolute diethyl ether (0.25 M solution) in a heat-dried 500 ml three-neck flask under an argon atmosphere, and 1 equivalent of 1-methoxy-2-methyl-1-trimethylsiloxypropene (C. Ainsworth, F. Chen, Y.-N. Kuo, J. Organomet. Chem. 1972, 46, 59-71) is added. After addition of 1 equivalent (where appropriate 3 equivalents) of boron trifluoride-diethyl ether complex, the mixture is heated to reflux for 24 hours. After the reaction mixture has cooled it is washed once each with 1N sodium hydroxide solution, water and saturated brine. The organic phase is dried over magnesium sulfate. Filtration and removal of the solvent are followed by purification of the crude product by column chromatography (silica gel: cyclohexane/ethyl acetate 9:1).

### Example 14A

### Methyl 2,2-dimethyl-3-(3-nitrophenyl)-3-oxopropanoate

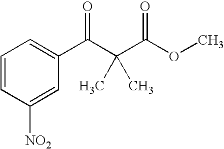

4.93 g (25% of theory) of product are obtained starting from 10 g (53.9 mmol) of 3-nitrobenzoyl chloride with 9.40 g (53.9 mmol) of 1-methoxy-2-methyl-1-trimethylsiloxypropene and 7.65 g (53.9 mmol) of boron trifluoride-diethyl ether complex.

HPLC (method 3): Rₜ=4.49 min

MS (DCI): m/z=269 (M+NH₄)⁺

General Procedure [F]: Pyrazolone Syntheses

1 equivalent of the β-keto ester is heated to reflux together with 5 equivalents of hydrazine hydrate in ethanol (0.23 M solution) for 4 hours. The reaction product precipitates from the reaction solution or it is precipitated after removal of part of the solvent with water and cyclohexane. The precipitate is filtered off with suction, washed with diethyl ether and then dried in vacuo.

### Example 15A

### 4,4-Dimethyl-5-(3-nitrophenyl)-2,4-dihydro-3H-pyrazol-3-one

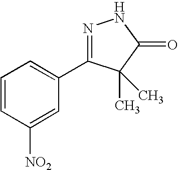

6.63 g (83% of theory) of product are obtained starting from 8.53 g (34 mmol) of methyl 2,2-dimethyl-3-(3-nitrophenyl)-3-oxopropanoate with 8.50 g (170 mmol) of hydrazine hydrate.

m.p.: 164.6° C.

HPLC (method 3): Rₜ=3.99 min

MS (DCI): m/z=251 (M+NH₄)⁺

General Procedure [G]: Catalytic Hydrogenation of the Nitro Group on the Aromatic System

20 mmol of the substance to be hydrogenated are dissolved in 100 ml of degassed methanol and then, under argon, 250 mg of palladium on activated carbon are added. Hydrogenation is carried out under a hydrogen atmosphere (atmospheric pressure) until a TLC check indicates complete conversion. This is followed by filtration with suction through kieselguhr, the filtrate is concentrated, and the residue is dried in vacuo and further processed without further purification.

### Example 16A

### 4,4-Dimethyl-5-(3-aminophenyl)-2,4-dihydro-3H-pyrazol-3-one

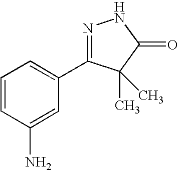

3.66 g (91% of theory) of product are obtained starting from 4.60 g (19.2 mmol) of 4,4-dimethyl-5-(3-nitrophenyl)-2,4-dihydro-3H-pyrazol-3-one.

HPLC (method 3): Rₜ=3.04 min

MS (ESIpos): m/z=204 (M+H)⁺

General Procedure [H]: Synthesis of the Hydrazinecarboxamides

1 equivalent of 2-[3-(acetylamino)benzoyl]hydrazinium chloride is introduced into dichloromethane (0.15 M solution) and stirred together with 2 equivalents of diisopropylethylamine and 1 equivalent of the appropriate isocyanate at room temperature for 16 hours. The resulting precipitate is filtered off with suction, washed with diethyl ether and dried in vacuo. The crude product is then directly reacted further.

### Example 17A

### 2-[3-(Acetylamino)benzoyl]-N-isopropylhydrazinecarboxamide

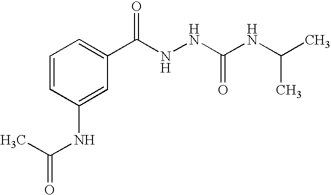

7.00 g (30.48 mmol) of 2-[3-(acetylamino)benzoyl]hydrazinium chloride are reacted with 7.88 g (60.96 mmol) of diisopropylethylamine and 2.59 g (30.48 mmol) of isopropyl isocyanate. The crude product is then directly reacted further.

HPLC (method 3): Rₜ=2.95 min

### Example 18A 2-[3-(Acetylamino)benzoyl]-N-cyclohexylhydrazinecarboxamide

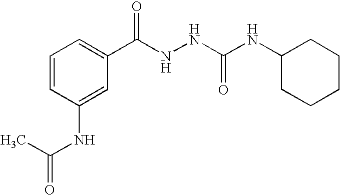

7.00 g (30.48 mmol) of 2-[3-(acetylamino)benzoyl]hydrazinium chloride are reacted with 7.88 g (60.96 mmol) of diisopropylethylamine and 3.82 g (30.48 mmol) of cyclohexyl isocyanate. The crude product is then directly reacted further.

HPLC (method 3): Rₜ=3.46 min

General Procedure [I]: Synthesis of the 3-Aminotriazolones

1 equivalent of the appropriate hydrazinecarboxamide is dissolved in 1 N sodium hydroxide solution (0.16 M solution), and 6.15 equivalents of sodium hydroxide are added. The mixture is stirred at 100° C. for 48 hours. The reaction solution is adjusted to pH 7 with hydrochloric acid, and the resulting precipitate is filtered off with suction, washed with water and dried in vacuo.

### Example 19A

### 5-(3-Aminophenyl)-4-isopropyl-2,4-dihydro-3H-1,2,4-triazol-3-one

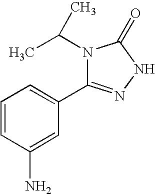

2.81 g (40% of theory) of product are obtained starting from 6.06 g (32.55 mmol) of 2-[3-(acetylamino)benzoyl]-N-isopropylhydrazinecarboxamide (crude) and 8.00 g (200.02 mmol) of sodium hydroxide in 200 ml of 1N sodium hydroxide solution.

HPLC (method 3): Rₜ=2.76 min

### Example 20A

### 5-(3-Aminophenyl)-4-cyclohexyl-2,4-dihydro-3H-1,2,4-triazol-3-one

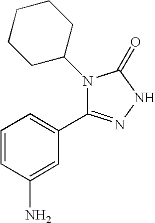

5.59 g (66% of theory) of product are obtained starting from 7.43 g (32.55 mmol) of 2-[3-(acetylamino)benzoyl]-N-cyclohexylhydrazinecarboxamide (crude) and 8.00 g (200.02 mmol) of sodium hydroxide in 200 ml of 1N sodium hydroxide solution.

HPLC (method 3): Rₜ=3.31 min

### DESCRIPTION OF THE FIGURE

FIG. 1: FIG. 1 shows the amino acid sequence of the wild-type HCMV UL86 protein (Acc. No. P16729, SEQ ID NO: 1)

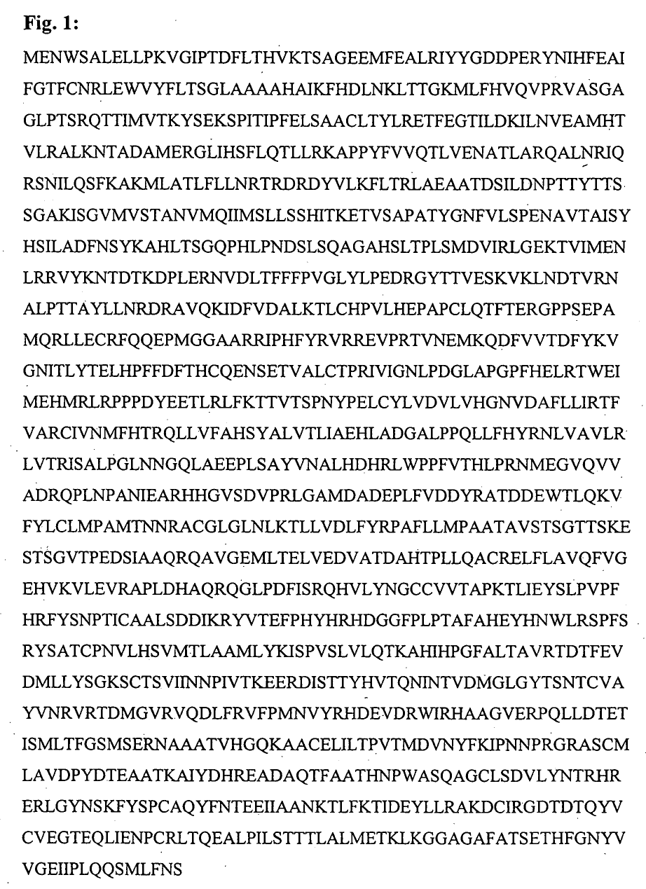

### EXEMPLARY EMBODIMENTS

### Example 1

### N-(2,4-Difluorophenyl)-N′-[3-(4,4-dimethyl-6-oxo-1,4,5,6-tetrahydro-3-pyridazinyl)phenyl]urea

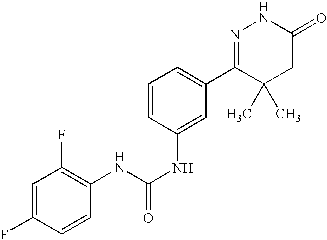

50 mg (0.23 mmol) of 6-(3-aminophenyl)-5,5-dimethyl-4,5-dihydro-3(2H)pyridazinone are mixed with 2 ml of abs. THF at room temperature, and then 71.4 mg (0.46 mmol) of 2,4-difluorophenyl isocyanate are added. The 6-(3-aminophenyl)-5,5-dimethyl-4,5-dihydro-3(2H)-pyridazinone does not initially dissolve completely. Only after addition of the isocyanate is a clear yellow solution obtained after a short time, but a white precipitate quickly separates out therefrom. The mixture is stirred overnight and then the precipitate is filtered off. The mixture is subsequently washed with diethyl ether, and the white solid is dried in vacuo. 46.4 mg (54% of theory) of producer are obtained.

m.p.: 213° C.

¹H-NMR (200 MHz, DMSO): δ=1.16 (s, 6H), 2.35 (s, 2H), 6.97-7.11 (m, 2H), 7.25-7.39 (m, 3H), 7.65 (s, 1H), 7.99-8.17 (m, 1H), 8.50 (s, br 1H), 9.12 (s, br 1H), 10.99 (s, 1H).

HPLC (method 3): Rₜ=4.12 min

MS (ESIpos): m/z=373 (M+H)⁺

### Example 2

### N-(2,5-Difluorophenyl)-N′-[3-(4,4-dimethyl-6-oxo-1,4,5,6-tetrahydro-3-pyridazinyl)-4-hydroxyphenyl]urea

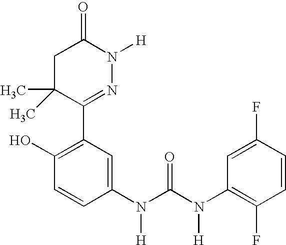

The synthesis takes place in analogy to example 1 from the appropriate precursors.

HPLC (method 3): Rₜ=4.00 min

MS (ESIpos): m/z=389 (M+H)⁺

### Example 3

### N-[4-({[(3-Chloro-4-fluorophenyl)amino]carbonyl}amino)phenyl]acetamide

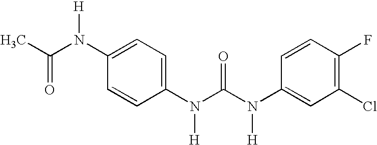

The compound can be purchased from Salor (Deisenhofen, Germany, Art. No. S90,580-1).

### Example 4

### N-[4-({[(3-Chloro-4-fluorophenyl)amino]carbonyl}amino)phenyl]pentanamide

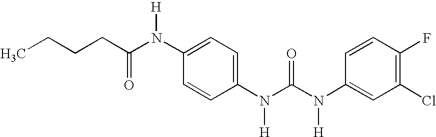

70.7 mg (0.894 mmol) of pyridine and 64.7 mg (0.536 mmol) of pentanoyl chloride are added to a solution of 100.0 mg (0.358 mmol) of example 11A in 5 ml of DMF. The mixture is stirred at RT for 18 h. After addition of water, a white precipitate separates out and is filtered off with suction and washed with water and pentane. The product is dried under high vacuum. 62 mg (80% of theory) of product are obtained.

R_{f} (cyclohexane/ethyl acetate 1:3): 0.44

HPLC (method 2): Rₜ=2.61 min

¹H-NMR (300 MHz, d₆-DMSO): δ=9.72 (s, 1H, NH), 8.79 (s, 1H, NH), 8.62 (s, 1H, NH), 7.83-7.74 (m, 1H, C₆H₃ClF), 7.55-7.32 (m, 4H, p-C₆H₄), 7.32-7.25 (m, 2H, C₆H₃ClF), 2.27 (t, 2H, CH₂), 1.58 (q, 2H, CH₂), 1.32 (sext, 2H, CH₂), 0.90 (t, 3H, CH₃).

### Example 5

### N-[3-({[(3-Chloro-4-fluorophenyl)amino]carbonyl}amino)phenyl]-1-butanesulfonamide

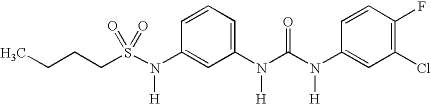

169.7 mg (2.145 mmol) of pyridine and 168.0 mg (1.073 mmol) of 1-butanesulfonyl chloride are added to a solution of 200.0 mg (0.715 mmol) of example 13A in a mixture of 2 ml of DMF and 5 ml of THF. The mixture is stirred at RT over night. No precipitate separates out after addition of water. The mixture is concentrated in vacuo. The product is purified by HPLC (RP18 column; mobile phase: acetonitrile/water, gradient 15:85->85:15). 51 mg (18% of theory) of product are obtained.

R_{f} (cyclohexane/ethyl acetate 1:2): 0.51

HPLC (method 1): Rₜ=4.28 min

¹H-NMR (300 MHz, d₆-DMSO): δ=9.74 (s, 1H, NH), 8.85 (s, 1H, NH), 8.78 (s, 1H, NH), 7.83-7.76 (m, 1H, C₆H₃ClF), 7.38-7.27 (m, 3H, m-C₆H₄, C₆H₃ClF), 7.24-7.18 (m, 2H, m-C₆H₄), 6.87-6.78 (m, 1H, m-C₆H₄), 3.08 (t, 2H, CH₂); 1.65 (q, 2H, CH₂), 1.35 (sext, 2H, CH₂), 0.83 (t, 3H, CH₃).

General Procedure [J]: Ureas

1 equivalent of the aniline is introduced into THF (0.14 M solution), and 1 equivalent of the appropriate isocyanate is added. The solution is shaken at room temperature for 1 hour. The solvent is removed in vacuo, and the product is purified by preparative HPLC (CromSil C 18, 250×30, flow rate: 50 ml/min, running time: 38 min, detection at 210 nm, gradient: 10% acetonitrile (3 min)->90% acetonitrile (31 min)->90% acetonitrile (34 min)->10% acetonitrile (34.01 min)).

Examples 6 and 7 can be prepared by general synthetic method [J].

| | | | MS | | |
|---|---|---|---|---|---|
| | | Molecular | (ESI+) | HPLC | HPLC |
| Example | Structure | weight | m/z | Rₜ [min] | method |
| 6 | 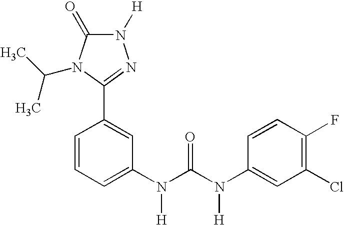 | 389.82 | 390 | 4.25 | 3 |
| 7 | 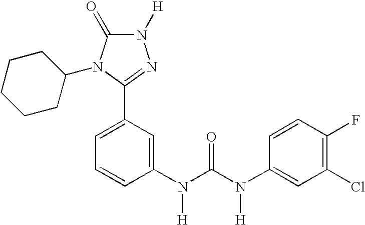 | 429.88 | 430 | 4.61 | 3 |

### Example 8

### N-(4-Chloro-2-methylphenyl)-N′-[3-(4,4-dimethyl-5-oxo-4,5-dihydro-1H-pyrazol-3-yl)phenyl]urea

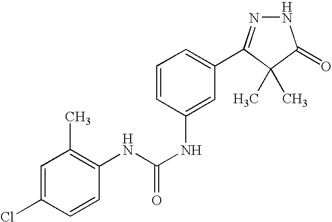

46.2 mg (0.28 mmol) of 4-chloro-2-methylphenyl isocyanate are mixed with a solution of 40 mg of 5-(3-aminophenyl)-4,4-dimethyl-2,4-dihydro-3H-pyrazol-3-one in 1 ml of ethyl acetate and 0.2 ml of tetrahydrofuran and stirred at room temperature overnight. The formation of a white precipitate is observed during this.

Workup: The reaction mixture is concentrated and the resulting residue is taken up in DMSO and then purified by RP-HPLC. 42 mg (58% of theory) of product are obtained in this way.

m.p.: 226.8° C.

HPLC (method 3): Rₜ=4.33 min

MS (ESIpos): m/z=371 (M+H)⁺

¹H-NMR (200 MHz, DMSO): δ=1.37 (s, 6H), 2.25 (s, 3H), 7.21 (dd, 1H), 7.28 (d, 1H), 7.35-7.46 (m, 3H), 7.88 (d, 1H), 8.02 (s br, 1H), 8.11 (s br, 1H), 9.23 (s br, 1H), 11.54 (s br, 1H)

### B. ASSESSMENT OF THE PHYSIOLOGICAL ACTIVITY

The in vitro effect of the compounds of the invention can be shown in the following assay:

Virus Growing:

Human cytomegalovirus (HCMV), DavisSmith (ATCC VR807) or AD169 (ATCC VR538) strain, is grown in vitro on human embryonic prepuce fibroblasts (NHDF cells). After the NHDF cells have been infected with a multiplicity of infection (M.O.I.) of 0.01, the virus-infected cells are harvested 5-10 days later and stored in the presence of minimal essential medium (MEM), 10% fetal calf serum (FCS) with 10% DMSO at −80° C. A cell-free stock is produced by removing only the cell culture supernatant and immediately freezing at −80° C. After serial ten-fold dilutions of the virus-infected cells or of the cell culture supernatant of the virus-infected cells (cell-free virus), the titer is determined on 24-well plates of confluent NHDF cells after vital staining with neutral red.

Anti-HCMV (Anti-Human Cytomegalovirus) Cytopathogenicity Tests

The test compounds are employed as 50 millimolar (mM) solutions in dimethyl sulfoxide (DMSO). Ganciclovir®, Foscarnet® and Cidofovir® are used as reference compounds. After addition of in each case 2 μl of the 50, 5, 0.5 and 0.05 mM DMSO stock solutions to 98 μl portions of cell culture medium in row 2 A-H for duplicate determinations, 1:2 dilutions are carried out with 50 μl portions of medium up to row 11 of the 96-well plate. The wells in rows 1 and 12 each contain 50 μl of medium. 150 μl of a suspension of 1×10⁴ cells (human prepuce fibroblasts [NHDF]) are then pipetted into each of the wells (row 1=cell control) and, in rows 2-12, a mixture of HCMV-infected and uninfected NHDF cells (M.O.I.=0.001-0.002), i.e. 1-2 infected cells per 1000 uninfected cells. Row 12 (without substance) serves as virus control. The final test concentrations are 250-0.0005 μM. The plates are incubated at 37° C./5% CO₂ for 6 days, i.e. until all the cells are infected in the virus controls (100% cytopathogenic effect [CPE]). The wells are then fixed and stained by adding a mixture of formalin and Giemsa's dye (30 minutes), washed with double-distilled water and dried in a drying oven at 50° C. The plates are then assessed visually using an overhead microscope (plaque multiplier from Technomara). The IC₅₀ values found for the substances of the invention are listed in table 1:

TABLE 1
Antiviral activity in vitro

| Example | IC50[μM] | SI |
|---|---|---|
| 1 | 1 | 125 |
| 2 | 0.4 | 325 |
| 3 | 0.5 | 40 |
| 4 | 0.08 | 750 |
| 5 | 0.14 | 70 |
| 6 | 0.4 | 150 |
| 7 | 1.5 | 30 |
| 8 | 0.6 | 30 |

Selection and Analysis of Resistant Mutants

For this purpose, NHDF cells are seeded in tissue culture vessels. 5×10³ cells per well are seeded in 96-well plates and infected with cell-free HCMV AD169 with an moi of 0.03. The infections are cultivated under substance pressure which corresponds to 10 times the IC₅₀ of the substance. Preferably, 30-100 96-well plates inoculated as described are made up. Wells showing a cytopathic effect (CPE) comparable to a virus infection without substance pressure are analyzed further, i.e. the contents of the well containing the viruses (cells and cell culture supernatant) are passaged on fresh cell cultures and cultivated further under substance pressure. Finally, viral stocks are produced as described above and frozen. Serial ten-fold dilution of the virus-infected cells or of the cell culture supernatant of the virus-infected cells is followed by determination of the titer on 24-well plates of confluent NHDF cells after vital staining with neutral red. Finally, the susceptibility to various substances (IC₅₀ determination) is determined as described above.

Determination of the UL86 Sequence

The HCMVAD169 mutants selected as described above are grown in vitro on human embryonic prepuce fibroblasts (NHDF cells) under substance pressure (10×IC₅₀ HCMVAD169). After infection of the NHDF cells with a multiplicity of infection (M.O.I) of 0.01, the virus-infected cells are harvested 5-10 days later, and the total DNA is isolated from these cells with the aid of established standard methods (e.g. phenol extraction and ethanol precipitation). Qiagen Sequencing Services (Qiagen, Hilden) determined, after amplification of the viral UL86 gene by PCR, the DNA sequence, which was then transcribed into protein sequence. Compared with the initial strain HCMV AD169, the differences shown in table 2 were detectable in the protein sequence of the major capsid protein (UL86) in the resistant viruses. The IC₅₀ values [μM] of various substances on these mutants compared with the initial strain are likewise indicated. (n.d.=not measured). The substances acting on UL86 show a greatly reduced activity with the UL86 mutants, whereas the activity of the DNA replication inhibitor ganciclovir as control is unchanged. Depending on the nature of the substance, certain mutations lead to various extents of resistance. The cases in which the IC₅₀ was found to be increased by >10× are shown emboldened and underlined.

The suitability of the compounds of the invention for the treatment of HCMV infections can be investigated in the following animal model:

TABLE 2
HCMV strains with mutations in UL86 and their sensitivity to various HCMV UL86 inhibitors compared with the wild-type HCMV AD169

HCMV AD169 clone with mutation in UL86

| IC 50 [μM] | Wild- | | | | | | | | | | | | |
|---|---|---|---|---|---|---|---|---|---|---|---|---|---|
| Substance | type | R435C | D441N | D563N | P586T | V601M | R682H | A689T | P1189T | Q1223R | A1226T | E1320Q | K1338N |
| Ganciclovir | 1 | 1 | 1 | 1 | 1 | 1 | 1 | 1 | 1 | 1 | 1 | 1 | 1 |
| 1 | 1 | 1.5 | 20 | >125 | 4 | 20 | 20 | 6 | >125 | 20 | >125 | >125 | >125 |
| 2 | 0.4 | 0.5 | 1.5 | 6 | 2 | 3 | 3 | 1 | >125 | 6 | >125 | 13 | >125 |
| 3 | 0.5 | >20 | >20 | >20 | >20 | >20 | >20 | >20 | >20 | >20 | >20 | >20 | >20 |
| 4 | 0.08 | >60 | 0.5 | 1 | 0.5 | 0.5 | 0.5 | 0.5 | 2 | 1 | 0.8 | 2 | >60 |
| 5 | 0.14 | 6 | n.d. | 1.4 | 0.8 | 1.5 | n.d. | 0.4 | n.d. | 1.4 | n.d. | 9 | >9 |
| 7 | 1.5 | 3 | n.d. | 15 | 7.6 | 12 | n.d. | 4.5 | n.d. | 12 | n.d. | 24 | 15 |
| 8 | 0.6 | 1.3 | n.d. | >21 | >21 | >21 | n.d. | >21 | n.d. | >21 | n.d. | >21 | >21 |

HCMV Xenograft Gelfoam® Model
Animals:

3-4-week old female immunodeficient mice (16-18 g), Fox Chase SCID or Fox Chase SCID-NOD or SCID beige, are purchased from commercial breeders (Bomholtgaard, Jackson, USA). The animals are housed under sterile conditions (including bedding and feed) in isolators.

Preparation of the Sponges, Transplantation, Treatment and Evaluation:

Collagen sponges 1×1×1 cm in size (Gelfoam®; from Peasel & Lorey, order No. 407534; K. T. Chong et al., Abstracts of 39^{th} Interscience Conference on Antimicrobial Agents and Chemotherapy, 1999, p. 439) are initially wetted with phosphate-buffered saline (PBS), the trapped air bubbles are removed by degassing, and then stored in MEM+10% FCS. 1×10⁶ virus-infected NHDF cells (infection with HCMV Davis or HCMV AD169 M.O.I.=0.03) are detached 3 hours after infection and added in a drop of 20 μl of MEM, 10% FCS, to a moist sponge. 12-13 hours later, the infected sponges are incubated with 25 μl of PBS/0.1% BSA/1 mM DTT with 5 ng/μl basic fibroblast growth factor (bFGF). For the transplantation, the immunodeficient mice are anesthetized with Avertin or a ketamine/xylazine/azepromazine mixture, the fur on the back is removed using a shaver, the epidermis is opened 1-2 cm, unstressed and the moist sponges are transplanted under the dorsal skin. The surgical wound is closed with tissue glue. 6 hours after the transplantation, the mice are treated for the first time (one treatment is given on the day of the operation). On subsequent days, oral treatment with the substance is carried out three times a day (7.00 h and 14.00 h and 19.00 h), twice a day (8 h and 18 h) or once a day (14 h) over a period of 8 days. The daily dose is for example 3 or 10 or 30 or 60 or 100 mg/kg of body weight, the volume administered is 10 ml/kg of body weight. The substances are formulated in the form of a 0.5% strength Tylose suspension with 2% DMSO or a 0.5% strength Tylose suspension. 9 days after transplantation and 16 hours after the last administration of substance, the animals are painlessly sacrificed and the sponge is removed. The virus-infected cells are released from the sponge by collagenase digestion (330 U/1.5 ml) and stored in the presence of MEM, 10% fetal calf serum, 10% DMSO at −140° C. Evaluation takes place after serial ten-fold dilutions of the virus-infected cells by determining the titer on 24-well plates of confluent NHDF cells after vital staining with neutral red. The number of infectious virus particles after the substance treatment compared with the placebo-treated control group is determined. The approximate results found for the substances of the invention are listed in table 3:

TABLE 3
Antiviral activity in vivo

| Example | ED50 [mg/kg/day] |
|---|---|
| 1 | 50 |
| 2 | 45 |
| 4 | 70 |

C. EXEMPLARY EMBODIMENTS OF PHARMACEUTICAL COMPOSITIONS

The compounds of the invention can be converted into pharmaceutical preparations in the following ways:

Tablet:

Composition:

100 mg of the compound of example 1, 50 mg of lactose (monohydrate), 50 mg of corn starch (native), 10 mg of polyvinylpyrolidone (PVP 25) (from BASF, Ludwigshafen, Germany) and 2 mg of magnesium stearate.

Tablet weight 212 mg. Diameter 8 mm, radius of curvature 12 mm.

Production:

The mixture of active ingredient, lactose and starch is granulated with a 5% strength solution (m/m) of the PVP in water. The granules are then dried and mixed with the magnesium stearate for 5 min. This mixture is compressed using a conventional tablet press (see above for format of the tablet). A guideline for the compressive force used for the compression is 15 kN.

Suspension which can be Administered Orally:

Composition:

1000 mg of the compound of example 1, 1000 mg of ethanol (96%), 400 mg of Rhodigel (xanthan gum from FMC, Pennsylvania, USA) and 99 g of water.

10 ml of oral suspension are equivalent to a single dose of 100 mg of the compound of the invention.

Production:

The Rhodigel is suspended in ethanol, and the active ingredient is added to the suspension. The water is added while stirring. The mixture is stirred for about 6 h until the swelling of the Rhodigel is complete.

1. A method for identifying compounds having anti-herpesvirus activity, characterized in that
(i) the major capsid protein or one or more fragments of the major capsid protein is/are brought into contact with test compounds, and
(ii) the binding of the test substances to the major capsid protein or fragments is measured and
(iii) the compounds which exhibit binding to the major capsid protein or fragments are selected.

2. The method as claimed in claim 1, where the herpesvirus is a human cytomegalovirus (HCMV).

3. A method for selecting compounds having anti-herpesvirus activity, characterized in that
(i) herpesviruses are brought into contact with test compounds,
(ii) resistant herpesviruses are selected,
(iii) the gene coding for the major capsid protein of these resistant herpesviruses is sequenced, and the resulting protein sequence of the major capsid protein is inferred,
(iv) the compounds with which resistant herpesviruses having one or more amino acid substitutions in the major capsid protein occur are selected.

4. The method as claimed in claim 3, where the herpesvirus is a human cytomegalovirus (HCMV).

5. The use of one or more substances which bind to the viral major capsid protein for producing a medicament for the treatment and/or prophylaxis of infections by herpesviruses.

6. The use as claimed in claim 5, where the herpesvirus comprises human cytomegaloviruses (HCMV).

7. The use of one or more substances identified by claim 3 for producing a medicament for the treatment and/or prophylaxis of infections by herpesviruses.

8. The use as claimed in claim 7, where the herpesvirus is the human cytomegalovirus (HCMV).

9. The use as claimed in any of claims 5 to 8, 23, or 24, characterized in that the substance(s) used therein permit the formation of non-infectious B capsids but not the formation of infectious C capsids.

10. The use of one or more substances which permit the formation of non-infectious B capsids but not the formation of infectious C capsids for producing a medicament for the treatment and/or prophylaxis of infectious by human cytomegaloviruses (HCMV).

11. The use as claimed in any of claims 5, 6, or 10, characterized in that viruses resistant to the substance used or to the substances used have one or more mutations in the amino acid sequence of the major capsid protein.

12. The use as claimed in any of claims 6, 8, 10, or 24, characterized in that one or more mutations in the UL86 protein at one or more of the following positions leads to resistance to the substances:
R435C, D441N, Y522C, D563N, P586T, V601M, R682H, A689T (cluster 1); P1189T, P1189S, Q1223R, A1226T, E1320Q, K1338N (cluster 2).

13. The use as claimed in claim 12, where there are one or more mutations between amino acids 400 and 700, and 1150 and 1370.

14. A method for inhibiting the replication of herpesviruses by substances which act on the major capsid protein.

15. The method as claimed in claim 14, where the formation of C capsids but not of B capsids is inhibited.

16. Method according to 14 or 15, where the herpesvirus is a human cytomegalovirus.

17. The use of substances which inhibit the replication of HCMVTowne only inadequately or not at all and inhibit the replication of wild-type HCMV strains for producing medicaments for the therapy and prophylaxis of HCMV infections.

18. A medicament produced by one of the methods as claimed in claims 5-8, 10, 13-15, 17, or 23-26.

19. A compound of the formula

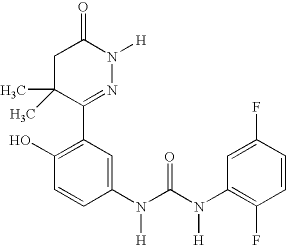

20. A compound of the formula

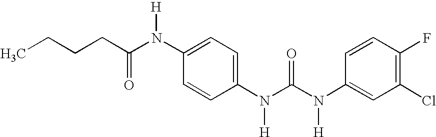

21. A compound of the formula

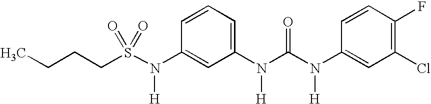

22. A compound of the formula 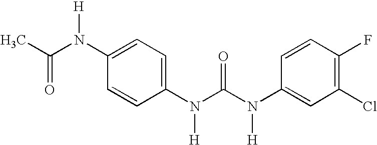
for the treatment and/or prophylaxis of diseases.

23. The use of one or more substances identified by claim 4 for producing a medicament for the treatment and/or prophylaxis of infections by herpesviruses.

24. The use as claimed in claim 23, where the herpes virus is the human cytomegalovirus (HCMV).

25. The use as claimed in claim 9, characterized in that viruses resistant to the substance used or to the substances used have one or more mutations in the amino acid sequence of the major capsid protein.

26. The use as claimed in claim 9, characterized in that one or more mutations in the UL86 protein at one or more of the following positions leads to resistance to the substances:
R435C, D441N, Y522C, D563N, P586T, V601M, R682H, A689T (cluster 1); P1189T, P1189S, Q1223R, A1226T, E1320Q, K1338N (cluster 2).

27. A medicament produced by one of the methods as claimed in claim 9.

28. A medicament produced by one of the methods as claimed in claim 11.

29. A medicament produced by one of the methods as claimed in claim 12.

30. A medicament produced by one of the methods as claimed in claim 16.


SEQUENCE LISTING (1 sequence)

SEQ ID NO: 1 - protein, 1370 aa, Human cytomegalovirus
MENWSALELLPKVGIPTDFLTHVKTSAGEEMFEALRIYYGDDPERYNIHFEAIFGTFCNRLEWVYFLTSGLAAAAHAIKFHDLNKLTTGKMLFHVQVPRVASGAGLPTSRQTTIMVTKYSEKSPITIPFELSAACLTYLRETFEGTILDKILNVEAMHTVLRALKNTADAMERGLIHSFLQTLLRKAPPYFVVQTLVENATLARQALNRIQRSNILQSFKAKMLATLFLLNRTRDRDYVLKFLTRLAEAATDSILDNPTTYTTSSGAKISGVMVSTANVMQIIMSLLSSHITKETVSAPATYGNFVLSPENAVTAISYHSILADFNSYKAHLTSGQPHLPNDSLSQAGAHSLTPLSMDVIRLGEKTVIMENLRRVYKNTDTKDPLERNVDLTFFFPVGLYLPEDRGYTTVESKVKLNDTVRNALPTTAYLLNRDRAVQKIDFVDALKTLCHPVLHEPAPCLQTFTERGPPSEPAMQRLLECRFQQEPMGGAARRIPHFYRVRREVPRTVNEMKQDFVVTDFYKVGNITLYTELHPFFDFTHCQENSETVALCTPRIVIGNLPDGLAPGPFHELRTWEIMEHMRLRPPPDYEETLRLFKTTVTSPNYPELCYLVDVLVHGNVDAFLLIRTFVARCIVNMFHTRQLLVFAHSYALVTLIAEHLADGALPPQLLFHYRNLVAVLRLVTRISALPGLNNGQLAEEPLSAYVNALHDHRLWPPFVTHLPRNMEGVQVVADRQPLNPANIEARHHGVSDVPRLGAMDADEPLFVDDYRATDDEWTLQKVFYLCLMPAMTNNRACGLGLNLKTLLVDLFYRPAFLLMPAATAVSTSGTTSKESTSGVTPEDSIAAQRQAVGEMLTELVEDVATDAHTPLLQACRELFLAVQFVGEHVKVLEVRAPLDHAQRQGLPDFISRQHVLYNGCCVVTAPKTLIEYSLPVPFHRFYSNPTICAALSDDIKRYVTEFPHYHRHDGGFPLPTAFAHEYHNWLRSPFSRYSATCPNVLHSVMTLAAMLYKISPVSLVLQTKAHIHPGFALTAVRTDTFEVDMLLYSGKSCTSVIINNPIVTKEERDISTTYHVTQNINTVDMGLGYTSNTCVAYVNRVRTDMGVRVQDLFRVFPMNVYRHDEVDRWIRHAAGVERPQLLDTETISMLTFGSMSERNAAATVHGQKAACELILTPVTMDVNYFKIPNNPRGRASCMLAVDPYDTEAATKAIYDHREADAQTFAATHNPWASQAGCLSDVLYNTRHRERLGYNSKFYSPCAQYFNTEEIIAANKTLFKTIDEYLLRAKDCIRGDTDTQYVCVEGTEQLIENPCRLTQEALPILSTTTLALMETKLKGGAGAFATSETHFGNYVVGEIIPLQQSMLFNS